SEQUENCE LISTING (2 sequences)

SEQ ID NO: 1 - protein, 6 aa, Artificial Sequence
  Synthetic
LYKYSD

SEQ ID NO: 2 - protein, 12 aa, Artificial Sequence
  Synthetic
GGSGGSGGSGGS

(19) United States
(12) Patent Application Publication
(10) Pub. No.: US 2023/0026666 A1
(43) Pub. Date: Jan. 26, 2023

(54) ENGINEERED BACTERIOPHAGE T4 NANOPARTICLES AS A POTENTIAL TARGETED ACTIVATOR OF HIV-1 LATENCY IN CD4+ HUMAN T-CELLS

(71) Applicant: The Catholic University of America, Washington, DC (US)

(72) Inventors: Venigalla B. RAO, Washington, DC (US); Himanshu BATRA, Washington, DC (US)

(21) Appl. No.: 17/736,181

(22) Filed: May 4, 2022

Related U.S. Application Data
(60) Provisional application No. 63/221,059, filed on Jul. 13, 2021.

(51) Int. Cl. C12N 15/86; C12N 5/0783; C12N 7/00

(52) U.S. Cl. CPC C12N 15/86; C12N 5/0636; C12N 7/00; C12N 2740/16023; C12N 2740/16122; C12N 2740/16222; C12N 2740/16042; C12N 2795/10023; C12N 2795/10022; C12N 2795/10042

(57) ABSTRACT

Described is an engineered viral particle programmed with T cell targeting specificity. The viral particles comprise: at least one viral vector, such as bacteriophage T4; and at least one CD4-binding protein displayed on the surface of the viral vector. Also described is a method of reactivate latent HIV-1 and cure patient with HIV-1 infection, using such an engineered viral particle.

### CROSS-REFERENCE TO RELATED APPLICATIONS

This application claims benefit of priority of U.S. Provisional Patent Application No. 63/221,059, entitled “Engineered Bacteriophage T4 Nanoparticles as a Potential Targeted Activator of HIV-1 Latency in CD4+Human T-cells,” filed Jul. 13, 2021. The entire contents and disclosures of these patent applications are incorporated herein by reference in their entirety.

### GOVERNMENT INTEREST STATEMENT

This invention was made with the United States government support under Grant Nos. AI111538 awarded by The National Institutes of Health (NIH). The government has certain rights in the invention.

### REFERENCE TO A “SEQUENCE LISTING”

The present application includes a Sequence Listing which has been submitted electronically in an ASCII text format. This Sequence Listing is named 109007-24118US01_sequence listing.TXT was created on Mar. 7, 2022, is 706 bytes in size and is hereby incorporated by reference in its entirety.

### BACKGROUND

### Field of the Invention

The present disclosure relates to generally to an engineered viral particle composition that reactivate latent HIV-1, as well as mechanisms and methods thereof.

### Background of the Invention

Bacteriophages (Phages) are present in diverse ecosystems ranging from the ocean to the human body.³¹ As the most abundant foreign organism in the human body, phages co-exist with human cells like other microflora such as bacteria and fungi.¹³ They home in a broad spectrum of organs, though prominently reside in the distal region of gut, either as prophage in a bacterial host or free virion embedded in the mucus layer.⁴ Some phages in the gut potentially translocate through epithelial monolayer into lamina propria and submucosal layer, where they can interact with the immune system.²⁴ While phages do not directly infect the human host, they are shown to potentially stimulate the immune system.¹⁰

Phage T4 decorated with pathogen epitopes mimic PAMPs (pathogen-associated molecular patterns) of natural viruses can stimulate strong innate as well as adaptive immune responses.³⁶ Induction of innate immune responses plays a critical role in HIV-1 infection and can reverse HIV-1 latency. Toll-Like Receptors (TLR) agonists such as TLR7, TLR9, TLR8, and TLR5 are shown to reactivate HIV-1 latent T-cells and used in the clinical trials for HIV-1 cure.³⁷

During HIV-1 infection, HIV enters the body and targets CD4+ T cells, and once inside the cell, the virus makes its way to the nucleus and inserts itself into the host cell's genomic DNA. Most of the infected CD4+ T-cells actively produce new viruses, but in a fraction of cells, the virus enters into a latent phase where it integrates into the human genome and remains transcriptionally silent. A collection of such cells carrying the HIV-1 “provirus” constitutes the latent “reservoir”.^{9,22} Although combinational antiretroviral therapy (cART) effectively controls the infection, it is not curative as it does not target the integrated proviruses. Hence, the viral rebound occurs by activation of a small pool of proviruses when treatment is discontinued.⁸ Thus, reducing or eliminating the latent reservoir is essential to achieve “remission” or “cure.” One of the best potential curative strategies is “shock and kill” which involves reversing the latent state by inducing HIV-1 transcription and subsequently killing the infected cells by the immune system.

Therefore, a method of reversing the latent state of HIV-1, without causing adverse effect in the patient is needed.

### SUMMARY

According to a first broad aspect, the present disclosure provides an engineered viral particle comprising: at least one viral vector; and at least one CD4 ligand, wherein the at least one CD4 ligand is displayed on the surface of the at least one viral vector.

According to a second broad aspect, the present disclosure provides an engineered viral particle comprising: at least one viral vector; and at least one HIV-1 envelope protein, wherein the at least one HIV-1 envelope protein is displayed on the surface of the at least one viral vector.

According to a third broad aspect, the present disclosure provides a method of activating a latent HIV-1 proviral genome comprising: adding an engineered viral particle of claim 1 to T cells containing latent HIV-1 proviral genome; and binding the engineered viral particle of claim 1 with T cells containing latent HIV-1 proviral genome.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

The accompanying drawings, which are incorporated herein and constitute part of this specification, illustrate exemplary embodiments of the invention, and, together with the general description given above and the detailed description given below, serve to explain the features of the invention.

FIG. 1 is a schematic diagram of the phage T4 structure according to one exemplary embodiment of the present disclosure.

FIG. 2 is a schematic diagram showing the components of bacteriophage T4-NPs according to one exemplary embodiment of the present disclosure.

FIG. 3 is a schematic diagram showing the preparation of phage T4-NPs according to one embodiment of the present disclosure.

FIG. 4 is a schematic diagram showing the mechanism of activation of the latent CD4+ T cells by the engineered T4 phage according to one exemplary embodiment of the present disclosure.

FIG. 5 is a figure showing the process of producing phage T4 empty capsids according to one exemplary embodiment of the present disclosure.

FIG. 6 is a graph showing the UV absorbance indicating elution of T4 heads according to one exemplary embodiment of the present disclosure.

FIG. 7 is a photo showing cryo-electron micrograph of purified T4 heads according to one exemplary embodiment of the present disclosure.

FIG. 8 is a graph showing the schematic representation of CD4DARPin (55.2)-Hoc fusion constructs according to exemplary embodiments of the present disclosure.

FIG. 9 is a reducing SDS-PAGE gel profiles showing bands of purified proteins according to one exemplary embodiment of the present disclosure.

FIG. 10 is a graph showing the production and purification of proteins according to one exemplary embodiment of the present disclosure.

FIG. 11 is a graph showing the binding of purified proteins to CD4 according to one exemplary embodiment of the present disclosure.

FIG. 12 is a graph showing the CD4 DARPin-Hoc binding fit curve with nonlinear regression according to one exemplary embodiment of the present disclosure.

FIG. 13 is a schematic diagram showing the route of target gene delivery by CD4DARP-T4-NPs according to one exemplary embodiment of the present disclosure.

FIG. 14 is a graph showing the specific binding of CD4 DARPin-Hoc to CD4 receptor 252 on T cells according to one exemplary embodiment of the present disclosure.

FIG. 15 is a graph showing the blocking of the binding of HIV Env Trimers to A3R5 cells by CD4DARPin Hoc fusion protein according to one exemplary embodiment of the present disclosure.

FIG. 16 is a photo showing the DNA packaging in T4 capsids according to one exemplary embodiment of the present disclosure.

FIG. 17 is a photo showing in vitro display of CD4DARPin-Hoc proteins according to one exemplary embodiment of the present disclosure.

FIG. 18 is a fluorescent photo showing the targeted delivery of eGFP DNA packaged in capsids decorated with or without CD4DARPin-Hoc according to one exemplary embodiment of the present disclosure.

FIG. 19 is a graph showing the expression of packaged and delivered luciferase gene quantified using luciferase assay according to one exemplary embodiment of the present disclosure.

FIG. 20 is a graph showing the flow cytometry data indicating binding of CD4DARP-T4-NPs to CD4+ T cells according to one exemplary embodiment of the present disclosure.

FIG. 21 is a schematic diagram showing the CD4DARPin-Hoc and eGFP-Soc co-display according to one exemplary embodiment of the present disclosure.

FIG. 22 is a photo showing the SDS-PAGE co-display gel profiles according to one exemplary embodiment of the present disclosure.

FIG. 23 is a photo showing the presence of a CD4 receptor according to one exemplary embodiment of the present disclosure.

FIG. 24 is a graph showing the specific binding of CD4 DARPin-Hoc to CD4 receptor on CD4+HEK293T cells according to one exemplary embodiment of the present disclosure.

FIG. 25 is a fluorescent photo showing the binding of CD4 DARPin-Hoc to CD4 receptor according to one exemplary embodiment of the present disclosure.

FIG. 26 is a photo showing the enlarged GFP/DAPI merged image of FIG. 25 according to one exemplary embodiment of the present disclosure.

FIG. 27 is a graph showing flow cytometry of single PBMCs from healthy human donors according to one exemplary embodiment of the present disclosure.

FIG. 28 is a graph showing flow cytometry of isolation of live PBMCs from healthy human donors according to one exemplary embodiment of the present disclosure.

FIG. 29 is a graph showing flow cytometry of isolation of CD3+ T cells according to one exemplary embodiment of the present disclosure.

FIG. 30 is a graph showing flow cytometry of separation of CD4+/CD4− cells according to one exemplary embodiment of the present disclosure.

FIG. 31 is a graph showing CD38 surface expression in CD3− T cells according to one exemplary embodiment of the present disclosure.

FIG. 32 is a graph showing CD38 surface expression in CD3+ T cells according to one exemplary embodiment of the present disclosure.

FIG. 33 is a graph showing CD38 surface expression in CD4+ T cells according to one exemplary embodiment of the present disclosure.

FIG. 34 is a graph showing CD38 surface expression in CD4− T cells according to one exemplary embodiment of the present disclosure.

FIG. 35 is a graph showing CD25 surface expression in CD3+ T cells according to one exemplary embodiment of the present disclosure.

FIG. 36 is a graph showing CD25 surface expression in CD4+ T cells according to one exemplary embodiment of the present disclosure.

FIG. 37 is a graph showing CD25 surface expression in CD4− T cells according to one exemplary embodiment of the present disclosure.

FIG. 38 is a graph showing the IL-2 secretion by T cells according to one exemplary embodiment of the present disclosure.

FIG. 39 is a graph showing the IFN-γ secretion by T cells according to one exemplary embodiment of the present disclosure.

FIG. 40 is a graph showing the IL-10 secretion by T cells according to one exemplary embodiment of the present disclosure.

FIG. 41 is a graph showing the TNF-α secretion by T cells according to one exemplary embodiment of the present disclosure.

FIG. 42 is a graph showing the IL-4 secretion by T cells according to one exemplary embodiment of the present disclosure.

FIG. 43 is a graph showing the IL-5 secretion by T cells according to one exemplary embodiment of the present disclosure.

FIG. 44 is a graph showing the IL-12 secretion by T cells according to one exemplary embodiment of the present disclosure.

FIG. 45 is a graph showing the IL-13 secretion by T cells according to one exemplary embodiment of the present disclosure.

FIG. 46 is a graph showing CCR5 surface expression in CD3+ T cells according to one exemplary embodiment of the present disclosure.

FIG. 47 is a graph showing CCR5 surface expression in CD4+ T cells according to one exemplary embodiment of the present disclosure.

FIG. 48 is a graph showing CCR5 surface expression in CD4− T cells according to one exemplary embodiment of the present disclosure.

FIG. 49 is a graph showing flow cytometry of single resting CD4+ T cells according to one exemplary embodiment of the present disclosure.

FIG. 50 is a graph showing flow cytometry of isolation of live resting CD4+ T cells according to one exemplary embodiment of the present disclosure.

FIG. 51 is a graph showing flow cytometry of isolation of CD3+CD4+ resting T cells according to one exemplary embodiment of the present disclosure.

FIG. 52 is a graph showing CD38 expression in purified CD4+ T cells according to one exemplary embodiment of the present disclosure.

FIG. 53 is a graph showing the quantification of surface expression of activation markers according to one exemplary embodiment of the present disclosure.

FIG. 54 is a graph showing the quantification of CD25 expression in resting T cells according to one exemplary embodiment of the present disclosure.

FIG. 55 is a graph showing the quantification of CD69 expression in resting T cells according to one exemplary embodiment of the present disclosure.

FIG. 56 is a graph showing the quantification of HLA-DR expression in resting T cells according to one exemplary embodiment of the present disclosure.

FIG. 57 is a graph showing the IL-2 secretion by resting T cells according to one exemplary embodiment of the present disclosure.

FIG. 58 is a graph showing the IFN-γ secretion by resting T cells according to one exemplary embodiment of the present disclosure.

FIG. 59 is a graph showing the IL-10 secretion by resting T cells according to one exemplary embodiment of the present disclosure.

FIG. 60 is a graph showing the TNF-α secretion by resting T cells according to one exemplary embodiment of the present disclosure.

FIG. 61 is a graph showing the IL-4 secretion by resting T cells according to one exemplary embodiment of the present disclosure.

FIG. 62 is a graph showing the IL-5 secretion by resting T cells according to one exemplary embodiment of the present disclosure.

FIG. 63 is a graph showing the IL-12 secretion by resting T cells according to one exemplary embodiment of the present disclosure.

FIG. 64 is a graph showing the IL-13 secretion by resting T cells according to one exemplary embodiment of the present disclosure.

FIG. 65 is a graph showing the construct design of gp140-soc fused trimers according to one exemplary embodiment of the present disclosure.

FIG. 66 is a graph showing the size-exclusion chromatography (SEC) result according to one exemplary embodiment of the present disclosure.

FIG. 67 is non-denaturing blue-NATIVE gel profiles showing the eluted fractions after SEC for FV100-soc according to one exemplary embodiment of the present disclosure.

FIG. 68 is reducing and non-reducing SDS-PAGE showing affinity chromatography-purified sample and size-exclusion chromatography trimeric fraction according to one exemplary embodiment of the present disclosure.

FIG. 69 is a graph showing the binding curve of PGT145 with gp140 and gp140-Soc according to one exemplary embodiment of the present disclosure.

FIG. 70 is a graph showing the binding curve of 8ANC195 with gp140 and gp140-Soc according to one exemplary embodiment of the present disclosure.

FIG. 71 is flow cytometry plots profiling showing the GFP expression in J-Lat cells when incubated with T4-NPs, CD4DARP-T4-NPs and controls NPs, and proteins according to one exemplary embodiment of the present disclosure.

FIG. 72 is a graph showing the quantification of flow cytometry plots shown in FIG. 71 according to one exemplary embodiment of the present disclosure.

FIG. 73 is a graph showing the binding specificity of CD4DARPin-Hoc and HIV-1 gp140-Soc fusion proteins according to one exemplary embodiment of the present disclosure.

FIG. 74 is a photo showing J-Lat 10.6 Full length cells infected with mCherry-Soc displayed or 484 CD4DARPin-Hoc and mCherry-Soc co-displayed T4-NPs according to one exemplary embodiment of the present disclosure.

FIG. 75 is a fluorescent photo showing the dose-response of CD4DARP-T4-NPs on reactivation of latent HIV-1 in J-Lat cells according to one exemplary embodiment of the present disclosure.

FIG. 76 is a fluorescent photo showing the time dependent reactivation of CD4DARP-T4-NPs in reactivation of latent HIV-1 in J-Lat cells according to one exemplary embodiment of the present disclosure.

FIG. 77 is a graph showing the Viral mRNA levels according to one exemplary embodiment of the present disclosure.

FIG. 78 is a graph showing the gag-derived p24 HIV protein in the cell culture supernatant assayed using an p24 ELISA according to one exemplary embodiment of the present disclosure.

FIG. 79 is a graph showing HIV-1 genomic RNA 474 levels in the cell culture supernatant according to one exemplary embodiment of the present disclosure.

FIG. 80 is a graph showing the effect of a PKC inhibitor on the reactivation of latent HIV-1 according to one exemplary embodiment of the present disclosure.

FIG. 81 is a graph showing the effect of a NFAT inhibitor on the reactivation of latent HIV-1 according to one exemplary embodiment of the present disclosure.

### DETAILED DESCRIPTION OF THE INVENTION

### Definitions

Where the definition of terms departs from the commonly used meaning of the term, applicant intends to utilize the definitions provided below, unless specifically indicated.

Unless defined otherwise, all technical and scientific terms used herein have the same meaning as is commonly understood to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present disclosure, the term “comprising,” the term “having”, the term “including,” and variations of these words are intended to be open-ended and mean that there may be additional elements other than the listed elements.

For purposes of the present disclosure, directional terms such as “top,” “bottom,” “upper,” “lower,” “above,” “below,” “left,” “right,” “horizontal,” “vertical,” “up,” “down,” etc., are used merely for convenience in describing the various embodiments of the present disclosure. The embodiments of the present disclosure may be oriented in various ways. For example, the diagrams, apparatuses, etc., shown in the drawing figures may be flipped over, rotated by 90° in any direction, reversed, etc.

For purposes of the present disclosure, a value or property is “based” on a particular value, property, the satisfaction of a condition, or other factor, if that value is derived by performing a mathematical calculation or logical decision using that value, property or other factor.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present disclosure, the term “bacterial viruses,” “bacteriophages” and “phages,” are used interchangeably. These terms refer to a virus or a viral particle that can infect bacteria.

For purposes of the present disclosure, the term “capsid” and the term “capsid shell” refer to the protein shell of a virus comprising several structural subunits of proteins. The capsid encloses the nucleic acid core of the virus.

For purposes of the present disclosure, the term “vector,” “vehicle,” and “nanoparticle” are used interchangeably. These terms refer to a virus or a hybrid viral particle that can be used to deliver genes or proteins.

For purposes of the present disclosure, the term “bind,” the term “binding” and the term “bound” refer to any type of chemical or physical binding, which includes but is not limited to covalent binding, hydrogen binding, electrostatic binding, biological tethers, transmembrane attachment, cell surface attachment and expression.

For purposes of the present disclosure, the term “nucleic acid” refers to polymers of nucleotides of any length, and include DNA and RNA. The nucleic acid bases that form nucleic acid molecules can be the bases A, C, G, T and U, as well as derivatives thereof. Derivatives of these bases are well known in the art. The term should be understood to include, as equivalents, analogs of either DNA or RNA made from nucleotide analogs. The term should also be understood to include both linear and circular DNA. The term as used herein also encompasses cDNA, that is complementary, or copy, DNA produced from an RNA template, for example by the action of reverse transcriptase.

For purposes of the present disclosure, the term “neck protein” and the term “tail protein” refer to proteins that are involved in the assembly of any part of the necks or tails of a virus particle, in particular bacteriophages. Tailed bacteriophages belong to the order Caudovirales and include three families: The Siphoviridae have long flexible tails and constitute the majority of the tailed viruses. Myoviridae have long rigid tails and are fully characterized by the tail sheath that contracts upon phage attachment to bacterial host. The smallest family of tailed viruses are podoviruses (phage with short, leg-like tails). For example, in T4 bacteriophage gp10 associates with gp11 to forms the tail pins of the baseplate. Tail-pin assembly is the first step of tail assembly. The tail of bacteriophage T4 consists of a contractile sheath surrounding a rigid tube and terminating in a multiprotein baseplate, to which the long and short tail fibers of the phage are attached. Once the heads are packaged with DNA, the proteins gp13, gp14 and gp15 assemble into a neck that seals of the packaged heads, with gp13 protein directly interacting with the portal protein gp20 following DNA packaging and gp14 and gp15 then assembling on the gp13 platform. Neck and tail proteins in T4 bacteriophage may include but are not limited to proteins gp6, gp25, gp53, gp8, gp10, gp11, gp7, gp29, gp27, gp5, gp28, gp12, gp9, gp48, gp54, gp3, gp18, gp19, gp13, gp14, gp15 and gp63.

For purposes of the present disclosure, the term “global activation” refers to the activation of signaling pathways that lead to the release of proinflammatory cytokines.

### Description

While the invention is susceptible to various modifications and alternative forms, specific embodiment thereof has been shown by way of example in the drawings and will be described in detail below. It should be understood, however that it is not intended to limit the invention to the particular forms disclosed, but on the contrary, the invention is to cover all modifications, equivalents, and alternatives falling within the spirit and the scope of the invention.

In one embodiment, CD4 receptor-targeting T4 nanoparticles (CD4DARP-T4-NPs) are used for activation of CD4+ T cells.

Phage T4 is a myovirus that infects the E. coli bacterium and belongs to the Order Caudovirales, representing the most abundant phage order in the intestine. FIG. 1 illustrates the phage T4 structure. Phage T4 has a large 120×86 nm prolate head (102) and a 140 nm-long contractile tail (108) to which six long tail fibers (116) are attached through a baseplate. The elongated T4 icosahedron is built with 930 molecules of the major capsid protein gp23* (“*” represents the cleaved mature form) (112), 55 copies of gp24* (104) at eleven of the twelve vertices, and 12 copies of the portal protein gp20 (106) at the unique twelfth vertex.⁷ The portal vertex is a ring structure with a central channel having a diameter of 3.5-4 nm through which DNA enters the capsid during packaging and exits during infection.³⁴ The surface of the T4 capsid is arrayed with two nonessential outer capsid proteins, Soc (small outer capsid protein) (110) (9.1 kDa; 870 copies per capsid) and Hoc (highly antigenic outer capsid protein) (114) (40.4 kDa; 155 copies per capsid).^{11,28,40} Soc is a tadpole-shaped molecule and binds at the quasi three-fold axes as a trimer. Each soc subunit acts as a “molecular clamp” by clasping two adjacent capsomers and, therefore, provides stability at high alkaline pH (pH 11). ²⁸ Hoc, on the other hand, is a 170 Å-long fiber containing a string of four Ig-like domains with the C-terminal domain bound to the center of each gp23* capsomer. One hundred and fifty-five symmetrically positioned Hoc fibers emanate from the T4 head.¹² Inside the capsid is a 171-kb linear dsDNA genome packaged by an ATP-powered pentameric molecular motor (gp17). The T4 phage tail is connected to the head via portal and neck proteins.

In one embodiment, the complex genetic program of phage T4 life cycle is manipulated to establish an in vitro system to generate engineered T4 nanoparticles.^{5,29,34}

In one embodiment, purified empty shells (head or capsid) lacking the outer capsid proteins Soc and Hoc, neck, and tail are filled with any linear DNA using the powerful T4 DNA packaging motor (gp17 ATPase) and the surface is arrayed with ˜1,025 molecules of foreign proteins fused to Hoc and Soc.

In one embodiment, the nanoparticles described above carrying a payload of genes and proteins are then delivered into human cells for various biomedical applications such as vaccine delivery and gene therapy.

In one embodiment, CD4-binding DARPin (Designed Ankyrin Repeat Proteins) are arrayed on T4 capsid lattice, creating nanoparticles that can efficiently target these nanoparticles (CD4DARPin-T4-NPs) and the associated payloads to CD4+ T cells.

In one embodiment, the nanoparticles displaying CD4-binding DARPin nearly completely titrate the CD4+ T cells from human peripheral blood mononuclear cells (PBMCs) and cause their activation.

In one embodiment, the activation of CD4+ T cells by the nanoparticles in the present disclosure is not global and does not lead to cytokine storm, or involve the classic protein kinase C (PKC) or nuclear factor of activated T cell (NFAT) pathways.

In one embodiment, the nanoparticles in the present disclosure cause reactivation of proviral genome resident in the HIV-1 latency reactivation model cell lines that leads to virion assembly and release.

In one embodiment, the engineered T4-NPs as HIV-1 viral mimics are used to target CD4+ T cells and cause activation, which can be exploited as a more deliberate “shock and kill” strategy for HIV-1 cure.

### Assembly of CD4DARPin-T4 Nanoparticles

The shock-and-kill strategy for HIV cure suggests that reactivation of virus transcription in CD4+ T cells containing a stably integrated and transcriptionally silent form of HIV-1 provirus, is the first essential step to eliminate the latent reservoir in ART-treated HIV-infected individuals.² In untreated patients, during the progression of HIV-1 infection, HIV-1 activates CD4+ T cells through its envelope glycoprotein interaction with the CD4 receptor. In cART suppressed patients, neither low viral copies (˜50 copies/ml or less) nor the soluble gp120 or gp140, shed from the viruses, are sufficient for activation.²¹

In one embodiment, the T4 nanoparticles (NPs) in the present disclosure can target CD4 receptor, mimicking HIV-1 interaction and activating CD4+ T cells (FIG. 1A).

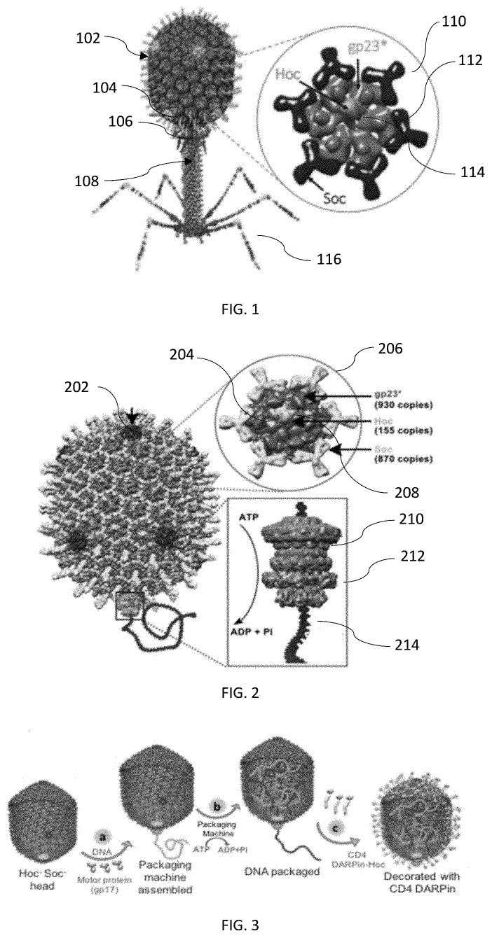

FIG. 2 illustrates the basic components of bacteriophage T4-NPs used in the present disclosure. As shown in FIG. 2, the structural model of phage T4 head (capsid) include pentameric gp24* vertices (202). The enlarged capsomer (hexamer) in FIG. 2 shows the arrangement of the major capsid protein gp23* (204), Soc trimers (206), and Hoc fiber (208) and enlarged portal vertex showing gp20 dodecamer (210), pentameric DNA packaging motor gp17 (212) and DNA to be packed (214).

In one embodiment, the T4-NPs in the present disclosure are decorated with CD4 binding DARPin (CD4DARPin) owing to its unique specificity and high affinity (1.5-83 nM) to CD4 receptor. CD4DARPin has been previously selected by in vitro evolution using DARPin libraries.³³

In one embodiment, T4-NPs were assembled by sequential incorporation of purified biomaterials to generate HIV-1 virus structural mimics. The sequential incorporation process is illustrated in FIG. 3. To prepare phage T4-NPs, gp17 motor proteins were assembled onto empty Hoc-Soc-phage T4 head (step a), empty Hoc-Soc-phage T4 head were packaged with DNA molecules by gp17 motor by using the energy from ATP hydrolysis (step b), and then CD4 targeting DARPin fused to Hoc were displayed on the heads (step c). In FIG. 3, the cut-out of the head shows both the exterior and the interior of the T4 head.

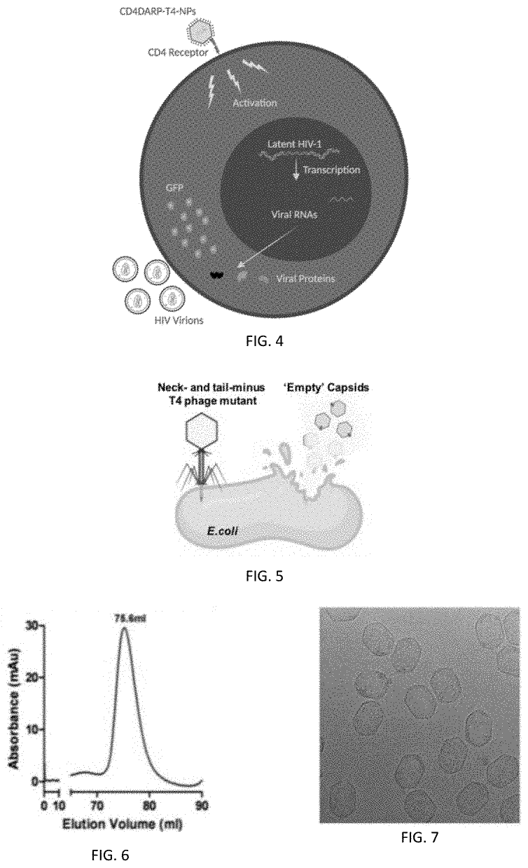

In one embodiment, the T4 phage capsid structure mimicking the molecular patterns associated with eukaryotic virus capsids and CD4 targeting activates the latent CD4+ T cells. FIG. 4 illustrates the mechanism of activation of the latent CD4+ T cells by the engineered T4 phage in the present disclosure. CD4 targeted phage T4-NPs mediate T cell activation and HIV-1 latency reversal. T4-NPs bind specifically to CD4 receptor, which leads to transcriptional activation of HIV-1 provirus and subsequent translation of viral mRNA to synthesize HIV proteins in addition to GFP which acts as a marker for activation. The virions assemble, bud and release from the activated cell.

In one embodiment, the T4-NPs were assembled, starting from an empty capsid shell isolated from a neck- and tail-minus T4 phage mutant-infected E. coli cells. FIG. 5 illustrated the process of producing phage T4 empty capsids. As shown in FIG. 5, T4 phage empty capsids can be purified by infection of non-suppressor E. coli with 10 am.13 am.Hocdel.Socdel phage. During the preparation of T4 heads as described in Examples, T4 heads were separated through a step gradient of CsCl (0.26 mg/mL at the top layer and 0.93 mg/mL at the bottom layer) and centrifuged at 40,000 rpm using SW55Ti rotor for 60 min. The elution peak of heads, further purified by binding to DEAE-Sepharose® ion-exchange chromatography (ÅKTA™ prime, GE® Healthcare). The bound heads were eluted with a NaCl gradient, and the heads were eluted at about 200 mM NaCl. The UV absorbance is plotted on the y-axis and elution volume on the x-axis, as shown in FIG. 6. FIG. 7 shows cryo-electron micrograph of purified T4 heads.

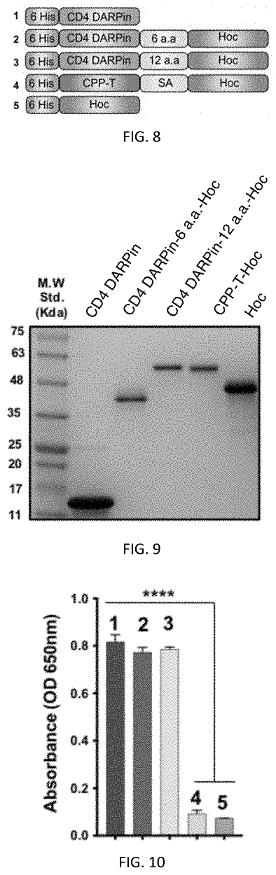

In one embodiment, to confer CD4+ T cells targeting ability, high-affinity CD4 DARPin fused to N-terminus of Hoc was displayed on the surface of the T4 capsids. The schematic representation of CD4DARPin (55.2)—Hoc fusion constructs is shown in FIG. 8. As shown in FIG. 8, the DARPin coding sequence was fused to the N-terminus of Hoc via 6-amino acid (6 a.a.) (LYKYSD) (SEQ ID No. 1) or 12-amino acid (12 a.a.) (GGSGGSGGSGGS) (SEQ ID No. 2) linkers. The unfused CD4DARPin, Hoc and cell penetration peptide (CPP)-T-Hoc were prepared as controls. A hexa-histidine purification tag (6 His) was fused to the N terminus of all proteins.

In one embodiment, the N-terminus of Hoc was used as it is projected away at ˜170 Å distance from the capsid wall, whereas the C-terminal domain of the Hoc fiber having the capsid binding site would be closest to the capsid.³² The CD4DARPin-Hoc fusion proteins; and CD4DARPin, Hoc, and cell penetration peptide (CPP)-TAT-Hoc that were overexpressed and purified from E. coli were used as controls. The successful production of purified proteins is shown in FIG. 9. The recombinant proteins were overexpressed in E. coli cells and purified from the lysates by nickel affinity chromatography using HisTrap column followed by Hi-load 16/60 Superdex® 200 gel filtration. In addition, the fusion partners CD4DARPin and Hoc acted as positive and negative controls respectively. The cell penetration peptide CPP-TAT is a 14-amino acid peptide rich in basic amino acids that was shown to facilitate passage of attached cargo molecules across the cell membrane.²³

In one embodiment, the fusion of Hoc and CD4DARPin have not affected the binding of CD4DARPin to CD4 receptor, as indicated by an ELISA performed using soluble human CD4 receptor with full-length extracellular domain (amino acids 1-370). The ELISA result is shown in FIG. 10. The data showed strong binding of CD4DARPin, CD4DARPin-Hoc fusion proteins, and JRFL-SOSIP-664 soluble trimers to soluble CD4 but not to the negative controls, Hoc and CPP-TAT-T4 Hoc. Error bars in FIG. 10 show S.D and P-value was determined using two-tailed, unpaired t-test, in which ****=P value<0.0001. The sample correspondence in FIG. 10 is as below:

| No. | Sample |
|---|---|
| 1 | CD4DARPin |
| 2 | CD4DARPin-6 a.a.-Hoc |
| 3 | CD4DARPin-12 a.a.-Hoc |
| 4 | CPP-T-Hoc |
| 5 | Hoc |

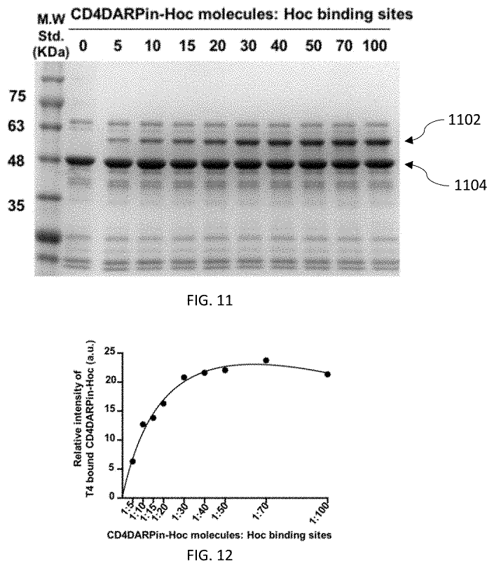

In one embodiment, display of CD4 DARPin-Hoc, Hoc, CD4 DARPin-12a.a-Hoc, and Hoc-T proteins on the T4 surface increased at increasing ratios of protein (CD4 DARPin-Hoc, Hoc, CD4 DARPin-12a.a-Hoc, and Hoc-T) to capsid Hoc binding sites. FIG. 11 provides an illustration of the quantification of displayed CD4 DARPin-Hoc at the ratios of CD4 DARPin-Hoc molecules to capsid Hoc ranging from 5:1 to 100:1. FIG. 11 shows the position of bound CD4 DARPin-Hoc (1102) and the position of gp23 of capsids (1104). The increase o displayed CD4 DARPin-Hoc at increasing ratios of CD4 DARPin-Hoc molecules to capsid Hoc binding sites is also confirmed by the CD4 DARPin-Hoc binding fit curve with nonlinear regression, as shown in FIG. 12. The density volumes of T4-bound CD4DARPin-Hoc were quantified by laser densitometry based on the SDS-PAGE results shown in FIG. 11.

### CD4DARPin T4-NPs Efficiently Target the CD4 Receptor on CD4+ T Cells

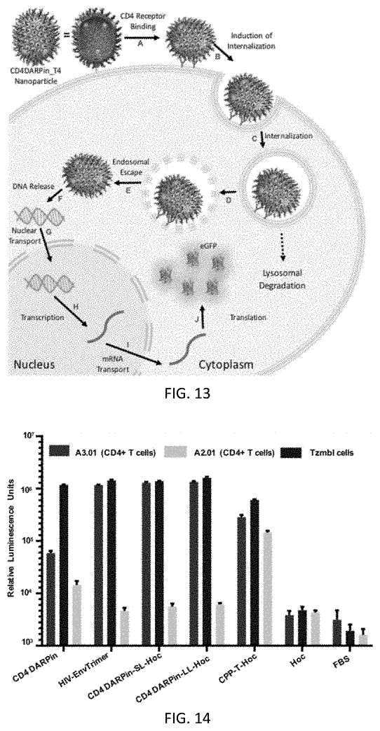

The targeting ability of CD4DARPin-T4-NPs was analyzed by its ability to deliver packaged plasmids encoding reporter gene. The route of target gene delivery by CD4DARP-T4-NPs is illustrated in FIG. 13. The NPs bind to cells through CD4 receptor and are internalized. The encapsidated DNA is released into the cytosol. In a preferred embodiment, the encapsidated DNA is gene encoding GFP. The DNA enters nucleus and undergoes transcription and expression of GFP.

In one embodiment, CD4 DARPin-Hoc protein variants recognize the CD4 receptor on the surface of T lymphocytes by using a modified cell-binding assay.⁶ The result of this test is shown in FIG. 14. For this assay, CD4DARPin test variants were coated on the wells in a 96-well plate and incubated with CD4-positive T cells (A3.01), CD4-negative T cells (A2.01), and CD4 overexpressing (Tzmbl) cells. After washing off the unbound cells, the CD4DARPin-bound cells were quantified using a luciferase-based, CellTiterGlo detection. As for the positive control, CPP-T-T4 Hoc, a nonspecific cell binder bound to both CD4-positive and CD4-negative cells. On the other hand, Hoc proteins used as a negative control showed no significant binding to any of the cells.

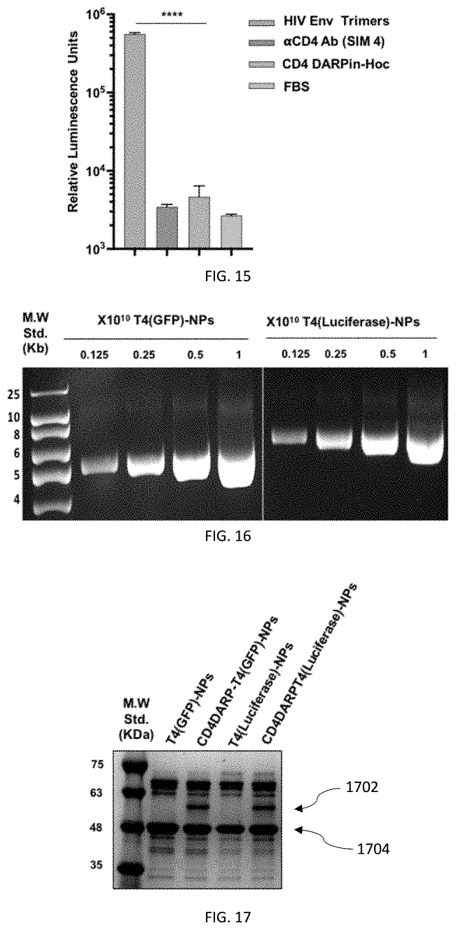

In one embodiment, CD4DARPin effectively blocked the binding of JRFL gp120 envelope protein (HIV Env Trimer) to CD4+ T cells (A3R5 cells) when tested in a cell binding assay, as shown in FIG. 15. These data demonstrate the specificity of binding of CD4DARPin-T4-NPs to CD4+ T cells in a manner similar to the binding of HIV-1 envelope protein.³³ CD4DARPin Hoc fusion protein blocked the binding of HIV Env Trimers to A3R5 cells (CD4+ T cells) in cell binding assay. A3R5 cells either untreated or treated with anti-CD4 antibody or CD4DARPin-Hoc were added to the wells and the extent of binding was quantified.

In one embodiment, the gp17 packaging ATPase motor protein was assembled at the portal vertex of T4 heads and the resultant packaging machine was used to package linearized DNAs in the presence of ATP. In a preferred embodiment, the linearized pAAV_mCherry or pAAV_Luciferase plasmid DNAs. The packaging reactions were terminated by the addition of DNase I that degrades the unpackaged DNA. The encapsidated and DNase I-resistant DNA was released by treatment with proteinase K and quantified by agarose gel electrophoresis as shown in FIG. 16.

In one embodiment, CD4DARPin-Hoc was displayed on the T4 heads by adding it to the reaction mixture and the unbound protein was removed by centrifugation and washing with buffer. FIG. 17 shows the successful display of CD4DARPin-Hoc. CD4DARPin-Hoc protein binding followed simple first-order kinetics.

In one embodiment, CD4DARPin-Hoc protein display can be controlled by varying the ratio of CD4DARPin-Hoc protein molecules to Hoc binding sites. At a ratio of 30:1, nearly all of the 155 Hoc binding sites were occupied, as shown in FIGS. 16 and 17. FIG. 17 shows the position of bound CD4 DARPin-Hoc (1702) and the position gp23 of capsids (1704).

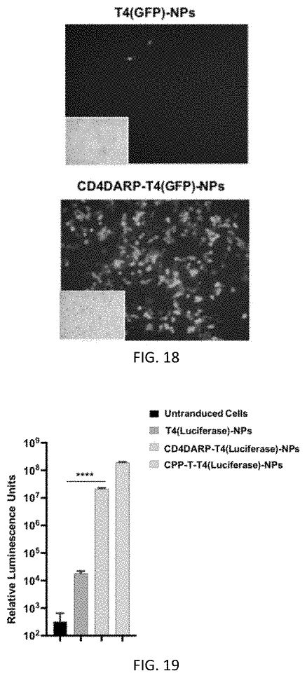

In one embodiment, the CD4DARPin-T4-NPs were able to target the delivery of the capsid-packaged eGFP and luciferase gene expressing plasmids into CD4+293T cells. As shown in FIG. 18, green florescence can be observed in T cells infected by T4 displayed with CD4DARPin-Hoc, but not T4 without CD4DARPin-Hoc. In FIG. 18. fluorescence and phase contrast micrographs of CD4+293T cells with CD4DARP-267 T4(GFP)-NPs is shown in the upper panel and T4(GFP)-NPs is shown in the lower panel. FIG. 19 shows the expression of packaged and delivered luciferase gene quantified using luciferase assay.

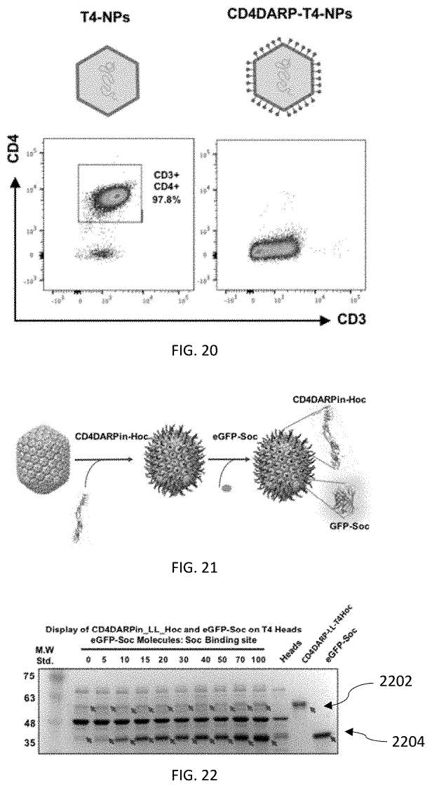

In one embodiment, the display of CD4DARPin-Hoc improves the efficiency of targeting T4-NPs. The CD4DARP-T4-NPs were incubated with freshly isolated human PBMCs followed by staining them with fluorochrome-conjugated anti-CD4 mAb. CD4DARP-T4-NPs efficiently bind CD4+ T cells in Human PBMCs human PBMCs, which were incubated with T4-NPs and CD4DARP-T4-NPs for 48 hours. Flow cytometry data shows that CD4+ T cells were undetectable with a CD4-specific antibody after treatment with CD4DARP-T4-NPs. Nearly all the CD4+ T cells (97.8%) infected with CD4DARP-T4-NPs could no longer be stained with anti-CD4 mAb when compared to the control cells treated with T4 heads lacking CD4DARPin, as shown in FIG. 20. In FIG. 20, error bars show the S.D. P-value determined using two-tailed, unpaired t-test, with ****=P value<0.0001. This experiment demonstrated that the CD4DARP-T4-NPs are highly efficient to target the CD4 280 receptor on the PBMCs that abrogated the binding of anti-CD4 mAb.

The high targeted binding efficiency of CD4DARP-T4-NPs was confirmed by an immunofluorescence assay by co-displaying the CD4DARPin-T4-NPs with eGFP-Soc and targeting these NPs to CD4 receptor-expressing 293T cells. The process of co-displaying is illustrated in FIG. 21. Strong GFP fluorescence encircled the periphery of these cells but not with the control NPs confirming the specificity of targeting of CD4DARP-T4-NPs to CD4+ cells.

FIG. 22 is an SDS-PAGE confirming the display of eGFP-Soc. About 4×10¹⁰ Hoc-Soc-heads were incubated with CD4DARPin-Hoc at 30:1 (CD4DARPin-Hoc molecule: Hoc binding sites). In one embodiment, increasing eGFP-Soc molecules to Soc-binding sites on the capsid at the ratio ranging from 0:1-100:1 increased the number of eGFP-Soc molecules displayed. The display of eGFP-Soc was carried out as described in Examples. After washing off the unbound protein, the samples were electrophoresed on a 4-20% gradient SDS-PAGE. FIG. 22 shows the positions of the CD4 DARPin-Hoc protein (2202) and eGFP-Soc (2204).

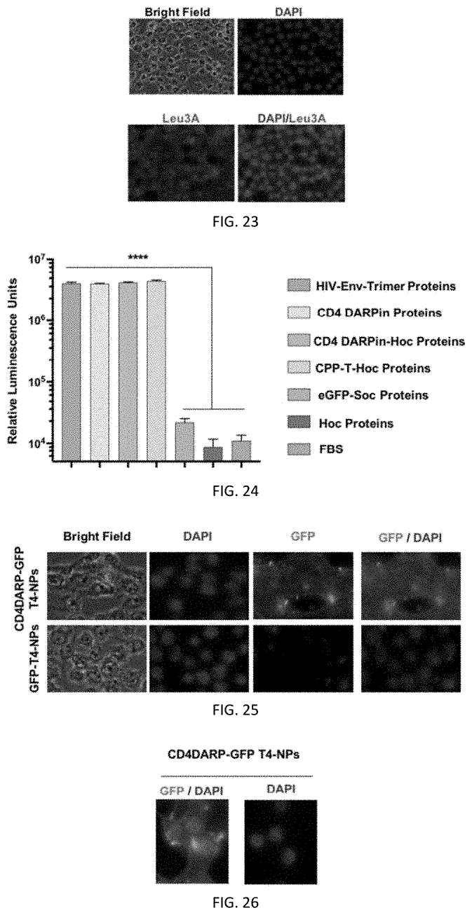

CD4+293T cells were grown on coverslips and fixed by CASIO methods, followed by co-staining with anti-CD4 antibody, Leu3a (Red), and nucleus stain, DAPI (Blue), as shown in FIG. 23. Red stain around the cell's periphery indicates the presence of a CD4 receptor on the surface.

The targeted binding of CD4DARP-T4-NPs to CD4+ T cells has also been confirmed by cell binding assay. Specific binding of CD4 DARPin-Hoc to CD4 receptor on CD4+HEK293T cells by cell binding assay is shown in FIG. 24.

The targeted binding of CD4DARP-T4-NPs to CD4+ T cells has also been confirmed by fluorescent staining. CD4+293T cells were grown on coverslips and fixed by CASIO methods, followed by incubating with either CD4DARPin-Hoc displayed or CD4DARPin-Hoc and eGFP co-displayed T4-NPs for 60 mins, followed by washing and fixing with DAPI-mount media and observation with fluorescence microscopy. The results are shown in FIG. 25, of which the images are 60× magnification. FIG. 26 shows an enlarged GFP/DAPI merged image of FIG. 25 at 100× magnification.

### The T4-NPs Activate Innate and Adaptive T Lymphocytes in Human PBMCs

In one embodiment, the T4-NPs in the present disclosure elicit a human T cell immune response.

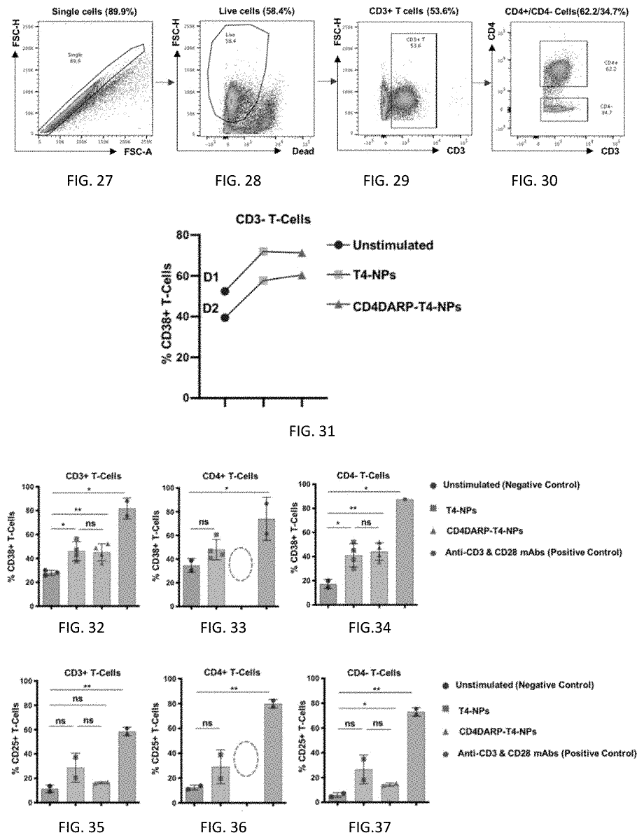

In one embodiment, the T cells can be activated by T4-NPs and express T cell activation marker. Freshly isolated human PBMCs from healthy donors were left unstimulated or were incubated with T4-NPs or the CD4DARP-T4-NPs. CD3/CD28 mAbs and/or PHA were used as positive controls for PBMCs activation. The activation status was determined by surface expression of CD25 and CD38, which were well-documented as late and early T cell activation markers respectively. After 48 hours of incubation, cells were co-stained for CD3, CD4 receptor, anti-CD25, and anti-CD38 and analyzed by flow cytometry. FIGS. 27-30 show the flow cytometry of PBMCs from healthy human donors left unstimulated or stimulated with the T4-NPs, CD4DARP-T4-NPs, and anti-CD3&CD28 antibodies.

CD25 is the alpha-chain of the IL-2 receptor and needs the beta-chain of the IL-2 receptor for the execution of IL-2 signaling.³⁰ CD38 is another well-characterized marker of immune activation in HIV infection that prolongs the proliferation and survival of CD4+ T cells.¹⁴ CD38 surface expression in T4-NPs and CD4DARP-T4-NPs treated cells showed the activation of innate immune cells (CD3− T cells), which include NK cells and dendritic cells.

In one embodiment, T4-NPs with or without CD4DARP displayed activate CD38 surface expression in CD3− T cells. FIG. 31 shows a comparison of CD3− T cells after different treatment. In FIGS. 31, D1 and D2 represents two different donors.

In one embodiment, a significant increase in surface expression of CD25 and CD38 was observed with T4-NPs and CD4DARP-T4-NPs, though profoundly less than the global activation shown by CD3/CD28 mAbs in CD3+ cells. FIGS. 32-34 show CD38 expression in cells with various treatment, while FIGS. 35-37 shows that of CD25. In FIGS. 33 and 36, CD4+ T cells were undetectable due to the targeted binding of CD4DARP-T4-NPs

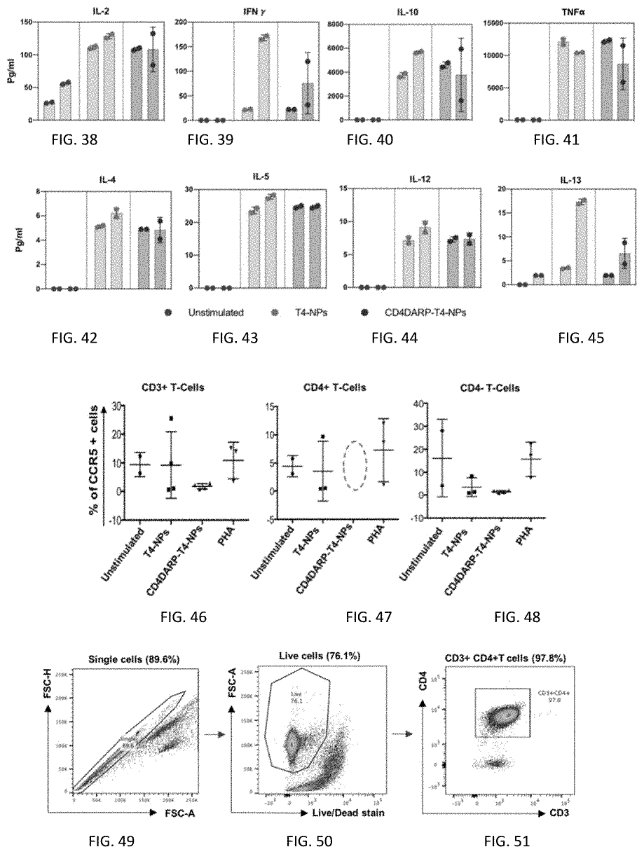

In one embodiment, the T4-NPs in the present disclosure activate T cells to secret cytokines. Profiling for secreted Th-1 (IL-2, IFN-γ, IL-10, TNF-α) and Th-2 (IL-4, IL-5, IL-12, IL-13) cytokines in cell supernatants reflected the immune cell activation by phage T4-NPs. In FIGS. 38-45, data of CD4+ T cells in PBMCs are shown as mean±SD. The data was analyzed using two-tailed unpaired t-test, with **=P value<0.005 and *=P value<0.05. Activation was observed in total CD3+ T cells and gated CD4+ and CD4− T cells, which represents the basal level of activation associated with T4-NPs. The observed activation of innate cells might indicate the transition from innate to adaptive direction of immune activation. The observed basal activation by T4-NPs might be due to the packaged double-stranded DNA containing unmethylated CpG motifs that are known to stimulate innate immune responses by interacting with Toll-like receptors (TLRs) such as TLR9 expressed on the endosomal surface of the antigen-presenting cells. ¹⁷

### CD4DARP-T4-NPs Non-Globally Activate Primary Resting CD4+ T Cells

Host responses to a viral vector may induce susceptibility of vector-specific T cells to HIV infection by upregulation of HIV-1 entry co-receptor, CCR5.³

In one embodiment, T4-NPs do not affect the susceptibility of vector-specific CD4 T cells to HIV infection, as evidence by analyzing surface expression of CCR5 in PMBCs using flow cytometry.

In one embodiment, neither T4-NPs nor CD4DARP-T4-NPs altered CCR5 expression in PBMCs, as shown in FIGS. 46-48.

CD4+ T-cells represent the major cell population of HIV latent reservoir. To determine if CD4DARP-T4-NPs can activate resting CD4+ T-cells, these cells from healthy human donor PBMCs were isolated by negative selection and their purity was established by flow cytometry (97.8% CD3+CD4+ T-cells). The flow cytometry result is shown in FIGS. 49-51, which confirms the purification of CD4+CD25− resting T cells from healthy human donors.

The purified cells were then either left unstimulated or stimulated with T4-NPs and CD4DARP-T4-NPs. CD3/CD28 mAbs and PMA mediated stimulations were used as positive controls. After 48 hours of incubation, CD38 surface expression was analyzed to determine the activation status of resting CD4+ T cells.

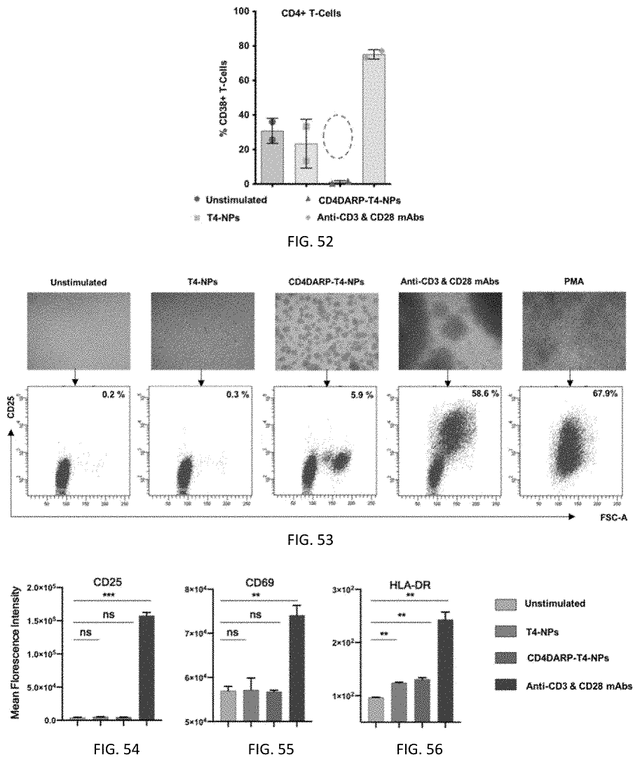

In one embodiment, no significant increase in CD38 expression in T4-NPs treated cells while CD4+ T cells were undetectable due to the targeted binding of CD4DARP-T4-NPs, as shown in FIG. 52.

In one embodiment, T cells are activated and show a characteristic clustering and increase in cell size upon stimulation by CD4DARP-T4-NPs. The clustering of the cells was observed under the microscope, in cells treated with CD4DARP-T4-NPs and anti-CD3/Cd28 mAbs or PMA (positive controls) but not with the T4-NPs treated cells or the untreated cells (negative control), as shown in the upper panels of FIG. 53. To further characterize the observed phenotypic activation, the surface expression of CD25, a global activation marker, was also determined. No significant increase in CD25 expression was observed. However, about 25% of the cells increased in size when treated with CD4DARP-T4-NPs, as evident from the dot-plots. As expected, anti-CD3/Cd28 mAbs activated cells showed a major increase in CD25 surface expression, as shown in the lower panels of FIG. 53. Similar phenotypical clustering and the increase in cell size have been reported previously when CD4+ T cells were treated with human immunodeficiency virus-1 (HIV-1) envelope protein gp120 that binds with human CD4 with high affinity.¹⁹

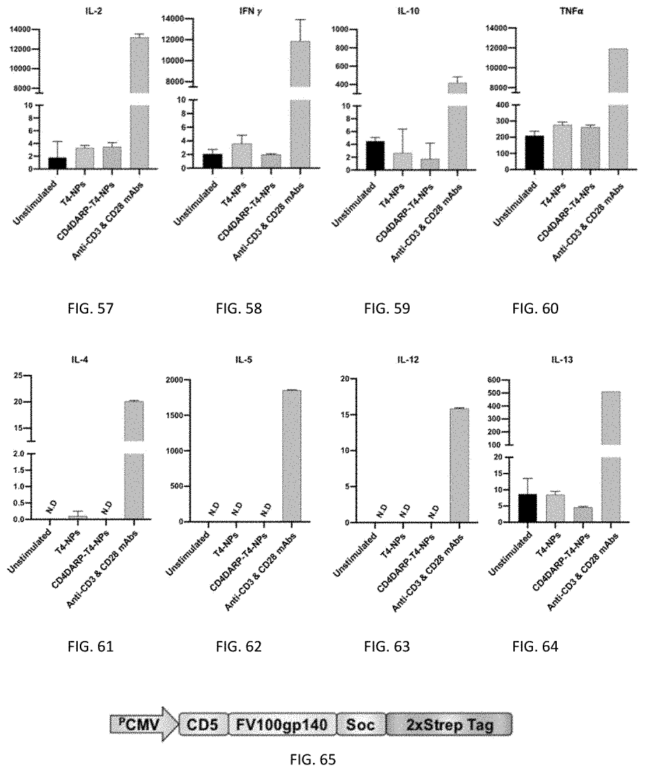

In one embodiment, CD4DARP-T4-NPs do not cause global T cell activation. While global activation of T cells can be destructive to host body, the non-global T cell activation is not. The surface expression of other activation markers such as CD25, CD69, HLA-DR, and CD38 were determined in the activated CD4+CD25− resting T cells isolated from healthy donors PBMCs by flow cytometry to probe the activation mechanism of this unexpected observation. No significant increase in the expression of any of these global T cell activation markers was observed in either T4-NPs or CD4DARP-T4-NPs, while both the positive controls showed substantial increases in the surface expression of these activation markers, as shown in FIGS. 54-56. In FIGS. 54-56, the data is presented as mean±SD, which was analyzed using two-tailed unpaired t-test, with ***=P value<0.001, **=P value<0.005, and *=P value<0.05. Similarly, the Th1 or Th2 specific cytokines were also not detected in culture supernatants of cells stimulated with either T4-NPs or CD4DARP-T4-NPs, as shown in FIGS. 57-64. In FIGS. 57-64, the data of resting CD4+ T cells is presented as mean±SD.

CD4DARP-T4-NPs and HIV-Env-T4-NPs Activate In Vitro Cell Model of Latent HIV-1 infection

In one embodiment, T4-NPs activate HIV latent cells. In a preferred embodiment, the HIV latent cells are J-Lat 10.6 full-length cells, a transformed cell line model of HIV-1 latency. The J-Lat cell line was originally developed by infecting Jurkat cells with a full-length HIV-1 vector having a frameshift mutation in the env gene and the gfp gene replacing nef. Nef gene encodes for NEF (Negative Regulatory Factor), a small 27-35 kDa myristoylated protein. Nef downregulates CD4 receptor and is involved in establishing a persistent state of infection by ensuring T cell activation. These cells don't produce viruses or viral proteins and are GFP-negative, indicating that they are transcriptionally silent and in the post-integration latency state. Therefore, expression of viral marker GFP would indicate activation of J-Lat cells.¹⁸

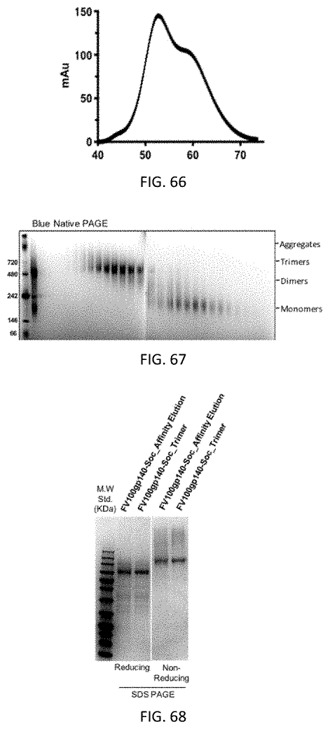

In one embodiment, two types of CD4 targeted phage T4-NPs were provided in the present disclosure, one displayed with CD4DARPin fused to Hoc and the other with a recombinant CRF AE HIV-1 gp140 (T/F100) fused to Soc.¹ HIV-1 gp140-Soc fusion protein construct was expressed in HEK293F cells and purified by Strep-Tactin affinity chromatography followed by size-exclusion chromatography. The design of gp140-soc fused trimers is illustrated in FIG. 65. FIG. 66 shows the result of size-exclusion chromatography (SEC), indicating purification of HIV-1 gp140-Soc fusion protein.

A series of biochemical analyses were performed after purification, such as Blue native PAGE, reducing and non-reducing PAGE to determine the proportion of trimers, nonspecific crosslinking and aggregation, and extent of furin cleavage. The blue native PAGE result is shown in FIG. 67, while the reducing and non-reducing PAGE is shown in FIG. 68. Both of these analyses confirmed the formation of trimers.

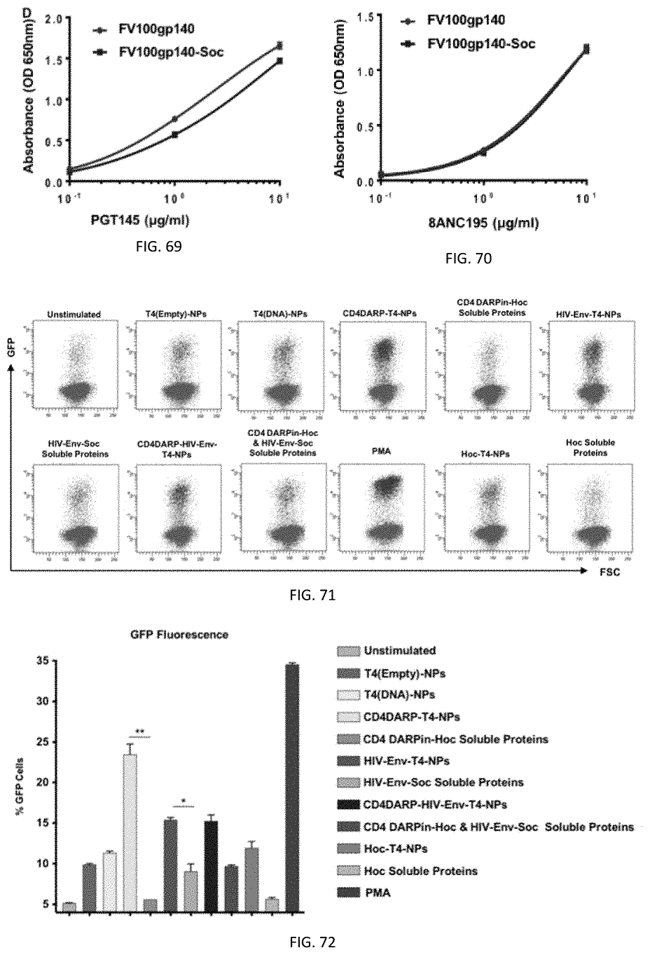

In one embodiment, gp140-Soc retained a “native-like” conformation of gp140. In addition, antigenicity analysis was done using PGT145 bNAb, which recognizes conformational epitopes at the apex of the closed trimer. The binding of PGT145 to gp140 and gp140-Soc is shown in FIG. 69. Being a quaternary Ab, PGT145 binds preferentially to the cleaved trimer in “closed” state but not to the “open” uncleaved trimers.³⁹ The 8ANC195 Ab was used as a control as it binds to both open and closed trimers, which is shown in FIG. 70. We found that the gp140-Soc fusion protein binds to the mAbs at similar efficiency as the gp140, suggesting that gp140-Soc retained a “native-like” conformation. In FIGS. 69 and 70, trimers were captured on the Strep-Tactin ELISA plates through the C-terminal Strep-Tag II at the constant concentration of 1 μg/ml for the assay. Each of FIGS. 69 and 70 shows the binding curve from three replicates at the indicated concentrations of the mAbs. No statistically significant difference in binding was observed between gp140-soc fusion and native gp140 trimer pairs at 10 μg/ml of the mAbs tested using unpaired two-tailed t test.

In one embodiment, the phage T4 capsid by itself can activate HIV-1 latent T cells. The T4-NPs, CD4DARP-T4-NPs and HIV-Env-T4-NPs, were incubated with J-Lat cells. After 48 hours, the phage T4 capsid by itself could show low but significant activation of HIV-1 latent T cells when compared to the untreated control as quantified by flow cytometry, as shown in FIGS. 71-72. FIG. 71 shows the flow cytometry result, while FIG. 72 shows the quantification of flow cytometry plots shown in FIG. 71. In FIG. 71, green and magenta dots in plots represents GFP positive and GFP negative cells respectively. X-axis represents intensity of GFP positive cells and Y-axis represents cell size (FSC). In FIG. 72, data are presented as means±S.D. of duplicate samples and represent two independent experiments.

The above result is consistent with the earlier observation that the T4-NPs can show low level of activation of human PBMCs and also T cells in general. This activation could be due to the surface structure of T4 capsid consisting of repetitive and symmetric disposition of capsid protein subunits that mimics the PAMPs (pathogen associated molecular patterns) present on human viral pathogens, or the residual ˜8 kb CpG phage DNA (TLR9 agonist) remaining inside the T4-NPs used in these experiments. However, the CD4DARP-T4-NPs that targeted the CD4 receptor showed far greater levels of J-Lat cell activation, 23.5% when compared to 9.9% with the T4-NPs. Furthermore, soluble (undisplayed) CD4DARPin-Hoc or JRFL HIV-1 gp140-Soc proteins at the same copy number could not show significant activation (data not shown). These observations suggest that the CD4 receptor-ligand displayed T4-NPs by mimicking the HIV-1 virion bound to J-Lat cells potentially through multipoint interactions that then led to efficient activation of the transcriptionally silent HIV provirus. This targeting specificity was demonstrated by several control experiments.

In one embodiment, CD4DARPin display enhances the targeting specificity of T4-NPs.

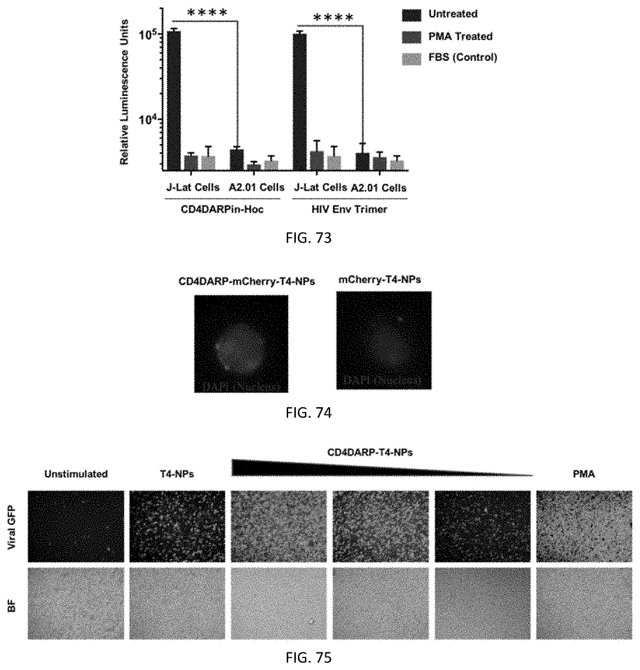

In one embodiment, CD4DARPin-Hoc and HIV-1 gp140-Soc fusion proteins bound specifically to J-Lat cells but not to CD4− T cells (A2.01) and PMA treated J-Lat cells which is known to downregulate the CD4 surface expression,²⁶ as shown in FIG. 73. In FIG. 73, P-value was determined using two-tailed, unpaired t-test, with ****=P value<0.0001.

In another embodiment, T4-NPs co-displayed with CD4DARPin-Hoc and mCherry-Soc but not mCherry-soc alone stained the J-Lat cells on the periphery. The fluorescent staining of the T cells is shown in FIG. 74, in which the Images are 60× magnification.

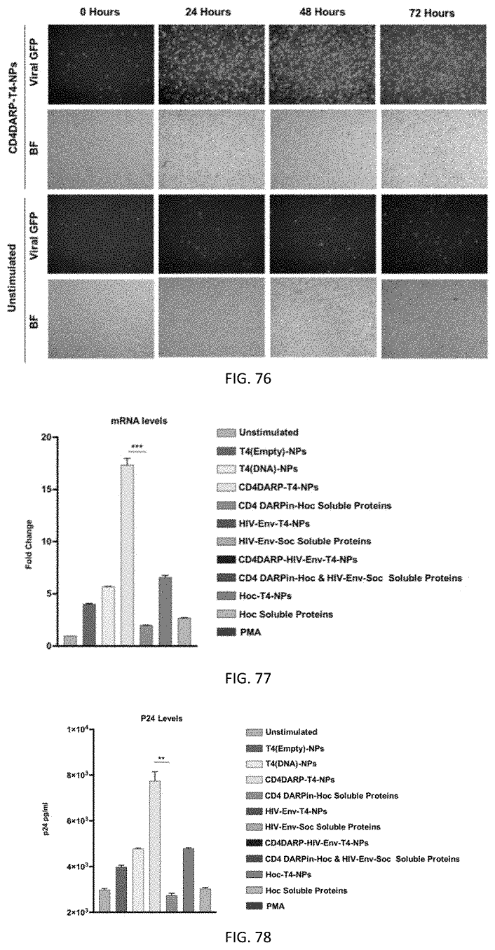

In one embodiment, CD4DARP-T4-NPs activate J-461 Lat cells in a dose and time-dependent manner as determined by the expression of viral GFP by fluorescence microscopy. FIG. 75 shows the dose-response of CD4DARP-T4-NPs on reactivation of latent HIV-1 in J-Lat cells, in which the effect of T4-NPs was determined by expression the GFP using fluorescence microscopy. FIG. 76 shows the time dependent reactivation of CD4DARP-T4-NPs in reactivation of latent HIV-1 in J-Lat cells, which is also determined by expression the GFP.

### CD4DARP-T4-NPs Reverse Latency in the In Vitro Cell Model of Latent HIV-1 Infection

In one embodiment, T4-NPs increase viral gene transcription, indicating activation of HIV-1 latent provirus in cells.

In a preferred embodiment, CD4DARP-T4-NPs increase viral gene transcription to a higher level, compared to T4-NPs.

Although the viral genome's GFP expression is an indicator of provirus activation, to confirm the effect of phage T4-NPs on the latent HIV-1, HIV-1 transcripts levels were examined by qPCR. The qPCR results correlated with GFP expression; the viral transcription was increased to 5.69-fold by T4-NPs, while CD4DARP-T4-NPs enhanced the transcription by 17.4-fold compared to the unstimulated cells. The viral gene transcription level after treatment with T4-NPs displaying different proteins is shown in FIG. 77. In FIG. 77, viral mRNA levels were quantified using real time RT-PCR and normalized to beta-actin mRNA levels. The results were compared to unstimulated samples and showed as fold change. Data are presented as means±S.D of duplicate samples. These results suggest transcriptional activation of the resident HIV-1 latent provirus in cells.

In one embodiment, T4-NPs increase assembly and release of HIV-1 virions, indicating activation of HIV-1 latent provirus in cells.

In a preferred embodiment, CD4DARP-T4-NPs increase assembly and release of HIV-1 virions to a higher level, compared to T4-NPs.

To further analyze the assembly and release of HIV-1 virions, the presence of HIV-1 virions in the culture supernatant were quantified by p24 ELISA. About 1.6-fold increase in HIV gag/p24 antigen was detected by T4-NPs while CD4DARP-T4-NPs enhanced it by 2.6-fold compared to unstimulated cells, as shown in FIG. 78.

Recently, it has been shown that latent cells can release viral proteins such as HIV-1 Gag protein without the actual viral particle release in an in vitro HIV mode1.²⁵

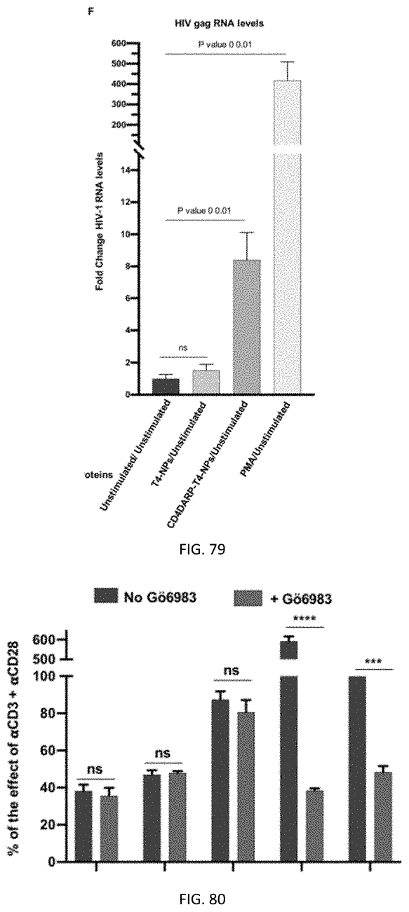

In one embodiment, phage T4-NPs increased release of the HIV-1 viral particles, instead of just the HIV Gag protein. To distinguish the release of the HIV-1 viral particles and the HIV Gag protein, the culture supernatant from the activated cells was concentrated and the viral RNAs were isolated. The copy number of HIV-1 genomic RNAs was determined by multiplex qPCR using a primer-probe set for HIV-1 gag and RCAS gag (internal control). An 8-fold increase in the HIV-1 RNA levels was observed in the culture supernatant of cells treated with CD4DARP-T4-NPs compared to supernatants of unstimulated cells, as shown in FIG. 79. In FIG. 79, the results are represented as fold change from unstimulated samples. Data are presented as means±S.D. of duplicate samples and represent two independent experiments.

In one embodiment, J-Lat cell activation leads to proviral transcription, and assembly and release of HIV-1 virions following the treatment with CD4DARP-T4-NPs.

### CD4DARP-T4-NPs Activate Latent HIV-1 Independent of PKC and NFAT Pathways

Physiologically, CD4 assists T-cell Receptor (TCR)'s communication with Antigen Presenting Cells (APCs). Specifically, CD4 receptor binds to MHC-II on the APCs mediating downstream signaling that ultimately leads to T-cell activation. PKCO is a major regulator in TCR signaling pathways.¹⁶

In one embodiment, CD4DARP-T4-NPs act to activate the latent HIV-1 in J-Lat cells, independent of PKC signaling pathway. Using the pan PKC inhibitor Gö6983, PKC inhibition completely suppressed PMA-induced activation of latent HIV-1 and reduced the anti-CD3 and anti-CD28 co-stimulatory response by ˜50%. However, no effect on the activation of latent HIV-1 by CD4DARP-T4-NPs was observed upon treatment with Gö6983, as shown in FIG. 80. These data suggest that CD4DARP-T4-NPs do not act via the PKC signaling pathway to activate the latent HIV-1.

NFAT, another essential transcription factor involved in the T cell activation, is also known to activate HIV-1 gene expression.²⁰

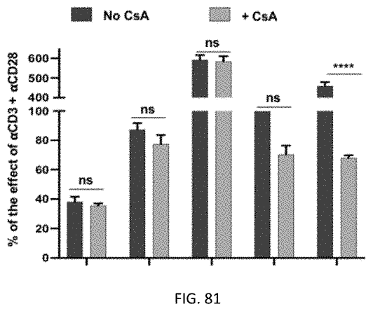

In one embodiment, CD4DARP-T4-NPs act to activate the latent HIV-1 in J-Lat cells, independent of NFAT signaling pathway. To assess the possibility of NFAT playing a role in J-lat activation, cyclosporin A (CsA), an inhibitor of the NFAT signaling pathway, was used. CsA treatment did not suppress the CD4-targeted CD4DARP-T4-NP reactivation of latent HIV-1, while it did inhibit the effect of ionomycin that activates T-cells via NFAT pathway, as shown in FIG. 81. In FIGS. 80-81, data are presented as mean±SD of duplicate samples from a representative experiment. P-value was determined using two-tailed, unpaired t-test, with ****=P value<0.0001, and ***=P value<0.001.

In one embodiment, the CD4DARP-T4-NPs mediated activation is not global activation as the PKC and NFAT pathways are two critical pathways involved in global T cell activation.

HIV remains a significant global health problem as nearly 38 million people worldwide are currently living with HIV-1 and ˜2 million new infections occur every year, but there is neither a vaccine nor a cure. The “shock-and-kill” strategy for HIV cure postulates that reactivation of virus transcription in CD4+ T cells containing a stably integrated and transcriptionally silent form of HIV-1 provirus is the first essential step to eliminate the latent reservoir in ART-treated HIV-infected individuals. Specifically activate these latent cells in a way that would not cause global activation remain as major hurdles for clinical implementation of this strategy.

In one embodiment, engineered bacteriophage T4 nanoparticles target the CD4+ T cells and induce a degree of HIV proviral reactivation without leading to global T cell activation.

HIV-1 uses multivalent display of its envelope glycoprotein to efficiently bind and activate CD4+ T-cells. In one embodiment, an HIV-1 viral mimic by displaying the CD4 ligand, CD4 DARPin, fused to Hoc on the capsid surface of a bacterial virus, the phage T4, is provided in the present disclosure.

In one embodiment, such CD4 DARPin-T4-NPs bind to CD4 receptor with high affinity and exquisite specificity, as evidenced by ELISA and cell binding assays. These nanoparticles emanating Hoc molecules at symmetrical positions of the capsid, with the CD4DARPin positioned at the tips of ˜170 Å flexible fibers are well-positioned to capture the CD4 receptor on T cells.

In one embodiment, CD4DARPin-T4-NPs have efficient targeting ability. First, the delivery of packaged reporter genes-expressing plasmids into T cells resulted in ˜67120-fold increase in luciferase activity when compared to control T4-NPs lacking the CD4DARPin-Hoc display. Second, remarkably, CD4+ T cells in human PBMCs were titrated out after treatment with CD4DARP-T4-NPs and hence became undetectable by a CD4-specific antibody. Indeed, the eGFP- or mCherry-Soc co-displayed CD4DARPin-T4-NPs generated a layer of fluorescence around the CD4+ cells demonstrating the targeting specificity of the phage T4 HIV-mimic.

In one embodiment, both the T4-NPs and CD4DARPin-T4-NPs increase the surface expression of CD38 activation marker in both CD3+ T-cells and CD3− non-T cells which include adaptive T cells and innate cell types, respectively.

In one embodiment, T4-NPs efficiently activate PBMC through innate cell types such as dendritic cells and macrophages.

In one embodiment, T4-NPs mediated activation could be explained by the presence of residual ˜8 kb DNA present in the capsid. It was recently reported that phages can mediate the activation of the human immune system through interaction of CpG in genomic DNA with TLR9 in the endosomes of macrophages, which when co-cultured with CD4+ T cells cause the activation.¹⁵ The activation of PBMCs by T4-NPs was also reflected by the release of Thl and Th2 cytokines.

In one embodiment, the CD4DARPin-T4-NPs or the NPs displayed with the HIV-1 gp140 trimers caused robust activation of J-Lat cells as determined by a series of assays including viral GFP expression, increased proviral transcription, and release of assembled virions by the activated cells. This observation is consistent with the finding that implicated in the activation of CD4+ T cells by calcium-mediated signaling upon gp120 binding without necessarily involving viral entry.²⁷ CD4 receptor on T cells plays a critical role in enhancing TCR mediated T cell activation by interacting with MHC class II molecules on antigen presenting cells (APCs). The CD4 receptor, on its cytoplasmic domain, interact with the src family tyrosine kinase p561ck which is involved in initiating TCR signaling. CD4 blockade by a soluble or membrane-bound gp120 is shown to be associated with activation of CD4+ T cells.

In one embodiment, activation by the phage T4 CD4DARPin-NPs does not lead to global T cell activation. This is an important distinction because many of the current shock-and-kill reagents investigated for HIV-1 cure lead to massive global activation that might lead to anaphylactic shock. This was evidenced by two observations. First, activation of J-lat cells does not activate PKC or NFAT pathways as demonstrated by the inhibitors of these pathways do not affect CD4DARPin-T4 mediated activation. PKC was documented to be a master regulator of multiple signaling pathways such as AP-1, NF-κB and ERK1/2. Also, NFAT mediated pathways are well documented in T cell activation. Bypassing the PKCO and NFAT pathways allows CD4DARP-T4-NPs to avoid the detrimental full-blown T cell activation.

In one embodiment, the CD4DARP-T4 NPs activation was also recapitulated in primary CD4+ T cells freshly isolated from human PBMCs. The cells showed no significant increase in the global activation markers such as CD69, CD25 and HLA-DR.

In one embodiment, cells treated with CD4DARP-T4 NPs showed clustering and increase in size. This phenotype has been shown previously to be associated with activation of T cells.³⁸ The clustering phenotype was also seen in ART suppressed patient derived CD4+ T cells when treated with CD4DARP-T4-NPs which suggest some kind of activation in CD4+ T cells, irrespective of HIV-1 infection, which is not global.

In one embodiment, the engineered phage T4 nanoparticles in the present disclosure serve as HIV-1 viral mimics and exhibit remarkable targeting specificity to CD4+ T cells.

In one embodiment, the engineered phage T4 nanoparticles in the present disclosure serve as a vehicle of delivering genes.

In one embodiment, the targeting ability of the engineered phage T4 nanoparticles in the present disclosure leads to T cell activation in a way that does not lead to global activation and cytokine storm.

In one embodiment, the targeting ability of the engineered phage T4 nanoparticles in the present disclosure leads to proviral HIV-1 genome reactivation and assembly and release of new virion particles in a HIV-latency model cell line but not in patient derived primary T cells.

In one embodiment, the targeted and selective activation of T cells hold promise for potential development of an effective HIV cure strategy and novel immune-therapeutics.

Having described the many embodiments of the present disclosure in detail, it will be apparent that modifications and variations are possible without departing from the scope of the invention defined in the appended claims. Furthermore, it should be appreciated that all examples in the present disclosure, while illustrating many embodiments of the invention, are provided as non-limiting examples and are, therefore, not to be taken as limiting the various aspects so illustrated.

### EXAMPLES

### Example 1

### Purification of 10-Amber 13-Amber Hoc (Highly Antigenic Outer Capsid Protein)-Del.Soc (Small Outer Capsid Protein)-Del Heads.

The 10-amber 13-amber hoc-del soc-del T4 phage mutant was constructed by standard genetic crosses and mutant heads were purified according to previously described protocols⁴¹. Briefly, E. coli P301 (sup-) cells (500 mL) infected with this mutant were lysed in 40 mL of Pi-Mg buffer (26 mM Na₂HPO₄/68 mM NaCl/22 mM KH₂PO₄/l mM MgSO₄, pH 7.5) containing 10 μg/mL DNase I and chloroform (1 mL) and incubated at 37° C. for 30 min. The lysate was subjected to two low-speed (6,000×g for 10 min) and high-speed (35,000×g for 45 min) centrifugations, and the final heads pellet was resuspended in 200 μL of Tris Mg buffer (10 mM Tris.HCl, pH 7.5/50 mM NaCl/5 mM MgCl₂) and purified by CsCl density gradient centrifugation. The major head band sedimented at about ⅓ from the bottom of a 5-mL gradient was extracted and dialyzed overnight against Tris.Mg buffer. The heads were further purified by DEAE-Sepharose® chromatography⁴¹. The peak heads fractions were concentrated and stored at ˜80° C.

### Example 2

### Cell Lines and Reagents

J-Lat full-length clone 10.6, ACH-2, A3.01, A2.01 and Tzmbl cells were obtained through NIH AIDS Research and Reference Reagent Program. Human PBMC, StemCell Technologies® Inc., CA (Catalog #70025). Phorbol 12-myristate 13-acetate, PMA (16561-29-8), PKC inhibitor, Gö6983, (133053-19-7), Cyclosporin A, CsA, (C3362), Ionomycin, (407952) were purchased from Sigma-Aldrich®. Monoclonal anti-CD3 (catalog no. 555336) and anti-CD28 (catalog no. 555725) antibodies were from BD® Biosciences. RPMI media without glutamine (Invitrogen®, 21870-076), OptiMEM (Gibco®, 31985-062), Pen Strep Glutamine (Gibco®-10378-016). Fetal Bovine Serum (Quality Biological®, Cat 110-001-101HI).

### Example 3

### Plasmid Construction

The plasmids pET-28b-Hoc and pET-28b-Cell Penetrating Peptide (CPP)-T-Hoc were constructed as previously described.^{32,35} pET-28b-CD4 DARPin (55.2) was constructed by fusing 6×-Histidine tag to the N-terminus of CD4 DARPin, 55.2 sequence from EMBL Nucleotide Sequence Database. The sequence was synthesized from Invitrogen® and amplified using following primers FW1 5′ ATATACCATGGGCAGCAGCCATCATCATCATC 3′ and BW1 5′TTAGGCTCGAGATTAAGCTTTTGCAGGATTTCAGCCAG 3′ having NcoI and XhoI cloning sites. The resulting fragment was purified by agarose gel electrophoresis, digested with appropriate restriction enzymes, and ligated with the gel-purified pET-28b vector DNA digested with the same restriction enzymes. For constructing pET28b-CD4DARPin-LYKYSD-Hoc and CD4DARPin-(GGGS)2GGSA-Hoc, two rounds of PCR were done. First round for the amplification of CD4 DARPin from pET-28b-CD4 DARPin_55.2 (FW1& BW2:) and Hoc from the pET-28b-Hoc clone (FW2: & BW3:). Full-length CD4 DARPin-Hoc was acquired by the second round of PCR using the FW1 and BW3 primers and then digested with NcoI and XhoI. The digested fragment was subcloned into the pET-28b vector. Plasmids pAAV-GFP and pAAV-mCherry were purchased from Cell Biolabs. All the constructed plasmids were sequenced to confirm correct fragment insertion (Retrogen®, CA).

### Example 4

### CD4 DARPin-Hoc Protein Expression and Purification

The recombinant proteins expressed in E. coli BL21 (DE3) RIPL cells were purified according to previously described protocols.⁴¹ Briefly, the BL21 (DE3) RIPL cells harboring the recombinant plasmids were induced with 1 mM IPTG for 2 h at 25° C. The cells were harvested by centrifugation (6,000×g for 15 min at 4° C.) and resuspended in 40 mL of HisTrap binding buffer (50 mM Tris.HCl, pH 8.0/20 mM imidazole/300 mM NaCl). The cells were lysed using French-press (Aminco®) and the soluble fraction containing the His-tagged fusion protein was isolated by centrifugation at 34,000×g for 35 minutes. The supernatant was loaded onto a HisTrap column (GE® Healthcare) and washed with 50 mM imidazole containing buffer, and the protein was eluted with 20-500 mM linear imidazole gradient. The peak fractions were collected and purified by size exclusion chromatography using Hi-Load 16/60 Superdex®-200 (prep-grade) gel filtration column (GE® Healthcare) in a buffer containing 25 mM Tris.HCl (pH 8.0) and 100 mM NaCl. The peak fractions were collected, flash frozen in Liquid N2 and stored at ˜80° C.

### Example 5

### Enzyme-Linked Immunosorbent Assay

ELISA plates (Evergreen Scientific®, CA) were coated with 0.1 μg of protein per well in coating buffer [0.05 M sodium carbonate-sodium bicarbonate (pH 9.6)] overnight at 4° C. After washing three times with PBS-T buffer, the plates were blocked with PBS-3% BSA buffer for 1 hour at 37° C. The recombinant soluble CD4 (NIH AIDS Reagent program, 4615) was added and incubated for 1 hour at room temperature. The bound fraction was detected by anti-CD4 Antibody Sim.2 (NIH AIDS Reagent program, 723) by ELISA using soluble CD4 at a dilution of 1:1000.

### Example 6

### Cell Binding Assay

Assays are performed as described previously.⁶ Briefly, purified CD4 DARPin variants and gp140 proteins were coated onto 96-well black, clear-bottom plate (Greiner®), 15 pmoles per well, for 1 hour. The wells were subsequently washed 3 times with blocking buffer (1 mM MnCl₂, 0.1 mM CaCl₂, 10 mM HEPES, 150 mM NaCl, and 10% FBS) and then incubated with the blocking buffer for 1 hour. Wells were then washed 3 times with wash buffer (1 mM MnCl₂, 0.1 mM CaCl₂, 10 mM HEPES, 150 mM NaCl, and 1% FBS). A3.01 (CD4+), A2.01 (CD4-), Tzmbl and J-Lat 10.6 full length cells (50 μl/well of 4×10⁶ cells per ml) were added in cell dilution buffer (wash buffer containing 5% FBS) and allowed to bind for 1 hour. Wells were then washed 5 times with wash buffer and the remaining bound cells were detected with the CellTiterGlo kit (Promega®) as per the manufacturer's instructions.

### Example 7

### In Vitro DNA Packaging and Protein Display of the T4 Head

For in vitro DNA packaging assays, each 20 μl of reaction mixture contained purified T4 heads (˜2×10¹⁰ particles), purified full-length gp17 (˜3 μM), and linearized DNA in packaging buffer [30 mM tris-HCl (pH 7.5), 100 mM NaCl, 3 mM MgCl₂, and 1 mM adenosine 5′-triphosphate (ATP)]. The mixture was incubated at 37° C. for 30 min, followed by benzonase nuclease addition and incubation at 37° C. for 20 min to remove excess unpackaged DNA. The encapsidated nuclease-resistant DNA was released by treatment with 50 mM EDTA, proteinase K (0.5 g/μl; Thermo Fisher Scientific®, MA), and 0.2% SDS for 30 min at 65° C. The packaged DNA was analyzed by 1% (w/v) agarose gel electrophoresis followed by staining with ethidium bromide, and the amount of packaged DNA was quantified using Quantity One software (Bio-Rad®, CA). The packaging efficiency was defined as the number of DNA molecules packaged per T4. In vitro protein display on the T4 head was assessed by the co-sedimentation described previously. Briefly, after encapsidating linearized DNA as described above, T4 heads were incubated with Soc- and/or Hoc-fused proteins at 4° C. for 45 min. The mixtures were sedimented by centrifugation at 30,000 g for 45 min, and unbound proteins in the supernatants were removed. After washing twice with PBS, the pellets were incubated at 4° C. overnight and then resuspended in PBS for SDS-polyacrylamide gel electrophoresis (SDS-PAGE) analysis or Opti-MEM for transduction. After Coomassie Blue R-250 (Bio-Rad®, CA) staining and destaining, the protein bands on SDS-PAGE gels were scanned and quantified by laser densitometry (Personal Densitometer SI; GE® Healthcare, Ill.). The densities of the Hoc, Soc, and gp23 bands were determined for each lane separately, and the copy numbers of bound Hoc or Soc fusion molecules per capsid were calculated using gp23 as the internal control (930 copies per capsid).

### Example 8

### Cell Transduction and the Detection of Gene Delivery

CD4+HEK293T cells were seeded in 24-well plates at 2.0×10⁵ cells per well in complete DMEM. After 24 hours, the cells were incubated with the T4, CD4DARPin-T4 vectors at different multiplicities of infection in antibiotic-free Opti-MEM for 6 hours. Thereafter, Opti-MEM was removed and replaced with complete DMEM. The cells were further incubated at 37° C. for an additional 48 hours. GFP transgene expression was observed by fluorescence microscopy (Carl Zeiss® Germany) at 48 hours after transduction. To analyze luciferase gene delivery into cells by T4 or CD4DARPin T4, luciferase activity was measured with the Luciferase Assay System (Promega®, WI) according to the manufacturer's instructions. Briefly, the growth medium was removed, and cells were rinsed with PBS buffer. After removing the wash buffer, 150 μl of passive lysis buffer was added to each well, followed by gentle shaking at RT for 20 mins. 20 μl of the cell lysate was then transferred to a 96-well white opaque plate and mixed with 80 μl of Luciferase Assay Reagent, and the luminescence signal was recorded using the GloMax-Multi Detection System (Promega®, WI).

### Example 9

### Cytokine Secretion Analysis

Fresh PBMCs (5×10⁵ cells per well) were left unstimulated or stimulated with T4-NPs, CD4DARP-T4-NPs, and anti-CD3 & anti-CD28 Abs for 48 hours at 37° C. Medium-treated PBMCs served as a negative control. After stimulation, cell-free supernatant was collected and analyzed by Bio-Plex Pro™ Human Cytokine Th1/Th2 Assay according to the manufacturer's instructions (Bio-Rad®). All tests were performed in duplicates, and mean values were calculated.

### Example 10

### Resting CD4+ T Cell Subset Isolation

Primary human CD4+ T cells were isolated from PBMCs using a Human resting CD4+ T Cells Isolation Kit I according to the manufacturer instructions (StemCell Technologies®). Briefly, PMBCs from liquid nitrogen were equilibrated at −80° C. for 12 hours, quickly thawed, and resuspended at 5×10⁷ cells/mL in PBS containing 2% FBS. To the sample, EasySep™ Human Resting CD4+ T Cell Isolation Cocktail, CD25 Depletion Cocktail, and Rapid Spheres were added, followed by incubation for 5 mins at room temperature. After incubation, the sample was top up to 2.5 ml with buffer containing PBS with 2% FBS and 1 mM EDTA, PBS should be without Ca²⁺ and Mg²⁺. The tube was placed into the magnet for 5 mins at RT and poured on in continuous motion into a new tube to get the enriched cell suspension. Purified CD4+ T cells were >97% pure as assessed by fluorescence-activated cell sorting.

### Example 11

### Measurement of the Reactivation of Latent HIV-1 in the Latent Cells

2.5×10⁵ J-Lat 10.6 full-length cells were resuspended in OPTIMEM media in flat bottom 24 well plate. The cells were then treated with the indicated concentrations and the number of activators or phage T4-NPs, respectively. After 6 hours, FBS was added to make up the final concentration to 10%. 2.5 μg/ml anti-CD3 and 1 μg/ml anti-CD28 monoclonal antibodies were used as positive controls. After 40-48 hours at 37° C., reactivation of latent HIV-1 was determined by quantifying the percentage of GFP+ cells using the FACS ARIA and analyzed using FlowJo® (BD® Biosciences) software. The percentage of GFP+ cells was calculated.

### Example 12

HIV-Specific mRNA Measurements

RNA was isolated using Direct-Zol® RNA MiniPrep Plus (Zymo Research®, Irvine, Calif.) and cDNA was synthesized by using iScript® cDNA Synthesis Kit (Bio-Rad® Laboratories, Hercules, Calif.). qRT-PCR was performed on Applied Biosystems StepOne™ Real-Time PCR System using iTaq™ Universal SYBR® green supermix (Bio-Rad® Laboratories, Hercules, Calif.) with HIV-1 mRNA primer set: 5′-GTGTGCCCGTCTGTTGTGTGA-3′, primer 2: 5′-GCCACTGCTAGAGATTTTCCA-3′ for and GAPDH primer set: 5′-AAGGTGAAGGTCGGAGTCAAC-3′ and 5′-GGGGTCATTGATGGCAACAATA-3′. The following cycling conditions were used for all qRT-PCR reactions: 95° C. for 10 min, 40 cycles at 95° C. for 15 sec and 60° C. for 1 min. Relative fold changes were calculated after normalization with GAPDH as a reference gene.

### Example 13

### p24 ELISA

Cell culture supernatant fluids were assayed for the Gag-derived p24 HIV protein using p24 ELISA kits (Zeptometrix®) by following the manufacturer's instructions. In principle, plate wells are pre-coated with anti-p24 gag (HIV-1) antibody. Virus particles in the culture supernatants upon lysis release p24 gag protein that binds to the immobilized antibody in the well. The captured p24 protein is then detected by a biotin conjugated anti-HIV-1 antibody. Streptavidin-Peroxidase binds to biotin and the amount of HIV-1 p24 antigen in the supernatant is finally quantified by colorimetry.

### Example 14

HIV RNA Isolation from Cell Supernatants

CD4+ T cells isolated from ART suppressed patients were left untreated or treated with T4-NPs, CD4DARP-T4-NPs, anti-CD3 and anti-CD28 mAbs, and PMA. The HIV RNA from the cell released free virus in the supernatant was extracted every 3 days for up to 9 days. The cryopreserved cell supernatants were thawed quickly at 37° C., 500 μl of the supernatant was diluted with Tris-buffered saline (TBS), and centrifuged at 21,100×g for 1 hour at 10° C. to pellet the cell-free virus. The supernatant was then removed, and the pellet was resuspended in 100 ml of 3M guanidinium hydrochloride (GuHCl) containing 50 mM Tris-HCl pH 7.6, 1 mM calcium chloride and 100 mg proteinase K and incubated at 42° C. for 1 hour. Next, 400 ml of 6 M guanidinium thiocyanate (GuSCN) containing 50 mM Tris HCl pH 7.6, 1 mM EDTA, and 600 mg/mL glycogen was added and incubated again at 42° C. for 10 minutes. After the second incubation, 500 ml of 100% isopropanol at room temperature was added to the guanidinium mixture, which was then invert mixed for 20 times and centrifuged at 21,000×g for 15 minutes at room temperature to pellet the RNA. Then, the supernatant was removed, and the pellet was washed with 70% ethanol. RNA pellets were air-dried for 5 mins and stored at −80° C. for the downstream assays.

### Example 15

cDNA Synthesis and qRT PCR for Patient Samples

Complementary DNA (cDNA) was synthesized from 10 μl of HIV RNA transcripts or extracted CA-RNA using Random hexamers and SuperScript® IV Reverse transcriptase according to manufacturer protocol (Thermo Fisher®). Next, a multiplexed qPCR master mix was made with a final concentration of 1× Lightcycler® 480 Probes Master Mix (Roche®, Switzerland), 600 nM forward and reverse primers, and 100 nM probe. A primer-probe set HIV-1 gag and RCAS gag was used. The copy number was determined from the standard curve generated from HIV RNA transcript standards that were diluted from 10×10⁶ to 1 copy/per well and assayed in triplicates for each run.

### Example 16

### Transfections of HIV-1 Env-Soc Trimers

HEK293F suspension cells were grown overnight to a density of 10⁶ cells/ml. Large-scale transfection in 1 L culture volume was done using FreeStyle MAX transfection reagent (Life Technologies®). Briefly, the cells were transfected with 1 μg of gp140-Soc plasmid DNA/million cells. As these clones are cleavage-sensitive, the cells were co-transfected with the furin plasmid at a gp140-Soc: furin plasmid DNA ratio of 3:1 to ensure near 100% cleavage. Plasmid DNAs and MAX reagent were diluted in OptiPRO® SFM medium, mixed and incubated for 10 min at room temperature. The mixture was then added to the HEK293F suspension cells. After 12 hours, the transfected cells were supplemented with 100 ml of fresh HyClone SFM4HEK293 medium (GE® Healthcare) and sodium butyrate⁴² solution (SIGMA-ALDRICH®; final concentration of 2 nM). On day 5, the supernatant was harvested by centrifuging the cells, and filtered using a 0.2 μm filter (Corning®, Inc.) for purification of the secreted protein.

### Example 17

HIV-1 gp140-soc Protein Purification

Secreted twin strep-tagged gp140-soc proteins in the harvested and filtered supernatant (1 L) were supplemented with protease inhibitor tablets (Roche® Diagnostics) to prevent protein degradation and 5 ml of BioLock® biotin blocking solution (IBA® Life Sciences GmbH) to mask the biotin present in the supernatant. Next, the supernatant was loaded onto a 1 ml Strep-Tactin column (Qiagen®) at a flow rate of 0.7 ml/min in the AKTA™ prime-plus liquid chromatography system (GE® Healthcare). Non-specifically bound proteins were washed off by passing at least 20 column volumes of wash buffer (50 mM Tris-HCl, pH 8, and 300 mM NaCl) until the absorbance reached the baseline level. Bound gp140-soc proteins were eluted with Strep-Tactin elution buffer (5 mM d-Desthiobiotin, 25 mM Tris-HCl, pH 8, and 150 mM NaCl) at a flow rate of 1 ml/min. Peak protein fractions were pooled and loaded onto the size-exclusion chromatography column using Hi-Load 16/60 Superdex®-200 (prep-grade) gel filtration column (GE® Healthcare) in a buffer containing 25 mM Tris.HCl (pH 8.0) and 100 mM NaCl. All the eluted fractions were collected and analyzed on BLUE Native PAGE and reducing/non-reducing SDS-PAGE for biochemical analysis.

### Example 18

### Strep-Tactin ELISAs

SEC purified HIV-1 Env-Soc trimers were tested for antigenicity using ELISA involving Microplates pre-coated with Strep-Tactin® (IBA® Life Sciences GmbH). Microplates were coated with 1 μg/ml SEC-purified gp140-Soc trimers in a volume of 100 μl/well of coating buffer (25 mM Tris-HCl, pH 7.6, 2 mM EDTA, and 140 mM NaCl) for 2 hours at room temperature. Followed by washing with PBST (0.05% Tween 20 in PBS) the trimers were incubated with serially diluted PGT145 and 8ANC195 antibodies for 1 hour at 37° C. The unbound Ab was washed off and the bound was detected by HRP-conjugated rabbit anti-human Ab (Santa Cruz Biotechnology®). Peroxidase substrate (TMB® microwell peroxidase substrate system, KPL®) was added and reaction was terminated by BlueSTOP solution (KPL®) and OD650 was measured (Molecular Devices®).

### REFERENCES

The following references are referred to above and are incorporated herein by reference:
1. Ananthaswamy, N., Fang, Q., AlSalmi, W., Jain, S., Chen, Z., Klose, T., Sun, Y., Liu, Y., Mahalingam, M., Chand, S., et al. (2019). A sequestered fusion peptide in the structure of an HIV-1 transmitted founder envelope trimer. Nat Commun 10, 873.
2. Archin, N. M., Sung, J. M., Garrido, C., Soriano-Sarabia, N., and Margolis, D. M. (2014). Eradicating HIV-1 infection: seeking to clear a persistent pathogen. Nat Rev Microbiol 12, 750-764.
3. Auclair, S., Liu, F., Niu, Q., Hou, W., Churchyard, G., Morgan, C., Frahm, N., Nitayaphan, S., Pitisuthithum, P., Rerks-Ngarm, S., et al. (2018). Distinct susceptibility of HIV vaccine vector-induced CD4 T cells to HIV infection. PLoS Pathog 14, e1006888.
4. Barr, J. J., Auro, R., Furlan, M., Whiteson, K. L., Erb, M. L., Pogliano, J Stotland, A., Wolkowicz, R., Cutting, A. S., Doran, K. S., et al. (2013). Bacteriophage adhering to mucus provide a non-host-derived immunity. Proc Natl Acad Sci USA 110, 10771-10776.
5. Black, L. W., and Rao, V. B. (2012). Structure, assembly, and DNA packaging of the bacteriophage T4 head. Adv Virus Res 82, 119-153.
6. Chand, S., Messina, E. L., AlSalmi, W., Ananthaswamy, N., Gao, G., Uritskiy, G., Padilla-Sanchez, V., Mahalingam, M., Peachman, K. K., Robb, M. L., et al. (2017). Glycosylation and oligomeric state of envelope protein might influence HIV-1 virion capture by alpha4beta7 integrin. Virology 508, 199-212.
7. Chen, Z., Sun, L., Zhang, Z., Fokine, A., Padilla-Sanchez, V., Hanein, D., Jiang, W., Rossmann, M. G., and Rao, V. B. (2017). Cryo-EM structure of the bacteriophage T4 isometric head at 3.3-A resolution and its relevance to the assembly of icosahedral viruses. Proc Natl Acad Sci USA 114, E8184-E8193.
8. Chun, T. W., Davey, R. T., Jr., Engel, D., Lane, H. C., and Fauci, A. S. (1999). Re-emergence of HIV after stopping therapy. Nature 401, 874-875.
9. Churchill, M. J., Deeks, S. G., Margolis, D. M., Siliciano, R. F., and Swanstrom, R. (2016). HIV 881 reservoirs: what, where and how to target them. Nat Rev Microbiol 14, 55-60.
10. Duerkop, B. A., and Hooper, L. V. (2013). Resident viruses and their interactions with the immune system. Nat Immunol 14, 654-659.
11. Fokine, A., Chipman, P. R., Leiman, P. G., Mesyanzhinov, V. V., Rao, V. B., and Rossmann, M. G. (2004). Molecular architecture of the prolate head of bacteriophage T4. Proc Natl Acad Sci USA 101, 6003-6008.
12. Fokine, A., Islam, M. Z., Zhang, Z., Bowman, V. D., Rao, V. B., and Rossmann, M. G. (2011). Structure of the three N-terminal immunoglobulin domains of the highly immunogenic outer capsid protein from a T4-like bacteriophage. J Virol 85, 8141-8148.
13. Gensollen, T., Iyer, S. S., Kasper, D. L., and Blumberg, R. S. (2016). How colonization by microbiota in early life shapes the immune system. Science 352, 539-544.
14. Glaria, E., and Valledor, A. F. (2020). Roles of CD38 in the Immune Response to Infection. Cells 9.
15. Gogokhia, L., Buhrke, K., Bell, R., Hoffman, B., Brown, D. G., Hanke-Gogokhia, C., Ajami, N. J., Wong, M. C., Ghazaryan, A., Valentine, J. F., et al. (2019). Expansion of Bacteriophages Is Linked to Aggravated Intestinal Inflammation and Colitis. Cell Host Microbe 25, 285-299 e288.
16. Isakov, N., and Altman, A. (2002). Protein kinase C(theta) in T cell activation. Annu Rev Immunol 20, 761-794.
17. Iwasaki, A., and Medzhitov, R. (2004). Toll-like receptor control of the adaptive immune responses. Nat Immunol 5, 987-995.
18. Jordan, A., Bisgrove, D., and Verdin, E. (2003). HIV reproducibly establishes a latent infection after acute infection of T cells in vitro. EMBO J 22, 1868-1877.
19. Kinet, S., Bernard, F., Mongellaz, C., Perreau, M., Goldman, F. D., and Taylor, N. (2002). gp120-mediated induction of the MAPK cascade is dependent on the activation state of CD4(+) lymphocytes. Blood 100, 2546-2553.
20. Kinoshita, S., Su, L., Amano, M., Timmerman, L. A., Kaneshima, H., and Nolan, G. P. (1997). The T cell activation factor NF-ATc positively regulates HIV-1 replication and gene expression in T cells. Immunity 6, 235-244.
21. Klasse, P. J., and Moore, J. P. (2004). Is there enough gp120 in the body fluids of HIV-1-infected individuals to have biologically significant effects? Virology 323, 1-8.
22. Maldarelli, F., Wu, X., Su, L., Simonetti, F. R., Shao, W., Hill, S., Spindler, J., Ferris, A. L., Mellors, J. W., Kearney, M. F., et al. (2014). HIV latency. Specific HIV integration sites are linked to clonal expansion and persistence of infected cells. Science 345, 179-183.
23. Milletti, F. (2012). Cell-penetrating peptides: classes, origin, and current landscape. Drug Discov Today 17, 850-860.
24. Nguyen, S., Baker, K., Padman, B. S., Patwa, R., Dunstan, R. A., Weston, T. A., Schlosser, K., Bailey, B., Lithgow, T., Lazarou, M., et al. (2017). Bacteriophage Transcytosis Provides a Mechanism To Cross Epithelial Cell Layers. mBio 8.
25. Pace, M. J., Graf, E. H., Agosto, L. M., Mexas, A. M., Male, F., Brady, T., Bushman, F. D., and O'Doherty, U. (2012). Directly infected resting CD4+ T cells can produce HIV Gag without spreading infection in a model of HIV latency. PLoS Pathog 8, e1002818.
26. Pelchen-Matthews, A., Parsons, I. J., and Marsh, M. (1993). Phorbol ester-induced downregulation of CD4 is a multistep process involving dissociation from p561ck, increased association with clathrin-coated pits, and altered endosomal sorting. J Exp Med 178, 1209-1222.
27. Perez, V. L., Rowe, T., Justement, J. S., Butera, S. T., June, C. H., and Folks, T. M. (1991). An HIV-1-infected T cell clone defective in IL-2 production and Ca2+ mobilization after CD3 stimulation. J Immunol 147, 3145-3148.
28. Qin, L., Fokine, A., O'Donnell, E., Rao, V. B., and Rossmann, M. G. (2010). Structure of the small outer capsid protein, Soc: a clamp for stabilizing capsids of T4-like phages. J Mol Biol 395, 728-741.
29. Rao, V. B., and Black, L. W. (2010). Structure and assembly of bacteriophage T4 head. Virol J 7, 356.
30. Ross, S. H., and Cantrell, D. A. (2018). Signaling and Function of Interleukin-2 in T Lymphocytes. Annu Rev Immunol 36, 411-433.
31. Salmond, G. P., and Fineran, P. C. (2015). A century of the phage: past, present and future. Nat Rev Microbiol 13, 777-786.
32. Sathaliyawala, T., Islam, M. Z., Li, Q., Fokine, A., Rossmann, M. G., and Rao, V. B. (2010). Functional analysis of the highly antigenic outer capsid protein, Hoc, a virus decoration protein from T4-like bacteriophages. Mol Microbiol 77, 444-455.
33. Schweizer, A., Rusert, P., Berlinger, L., Ruprecht, C. R., Mann, A., Corthesy, S., Turville, S. G., Aravantinou, M., Fischer, M., Robbiani, M., et al. (2008). CD4-specific designed ankyrin repeat proteins are novel potent HIV entry inhibitors with unique characteristics. PLoS Pathog 4, e1000109.
34. Sun, S., Kondabagil, K., Draper, B., Alam, T. I., Bowman, V. D., Zhang, Z., Hegde, S., Fokine, A., Rossmann, M. G., and Rao, V. B. (2008). The structure of the phage T4 DNA packaging motor suggests a mechanism dependent on electrostatic forces. Cell 135, 1251-1262.
35. Tao, P., Mahalingam, M., Marasa, B. S., Zhang, Z., Chopra, A. K., and Rao, V. B. (2013). In vitro and in vivo delivery of genes and proteins using the bacteriophage T4 DNA packaging machine. Proc Natl Acad Sci USA 110, 5846-5851.
36. Tao, P., Zhu, J., Mahalingam, M., Batra, H., and Rao, V. B. (2019). Bacteriophage T4 nanoparticles for vaccine delivery against infectious diseases. Adv Drug Deliv Rev 145, 57-72.
37. Tsai, A., Irrinki, A., Kaur, J., Cihlar, T., Kukolj, G., Sloan, D. D., and Murry, J. P. (2017). Toll-Like Receptor 7 Agonist GS-9620 Induces HIV Expression and HIV-Specific Immunity in Cells from HIV-Infected Individuals on Suppressive Antiretroviral Therapy. J Virol 91.
38. Vasiliver-Shamis, G., Cho, M. W., Hioe, C. E., and Dustin, M. L. (2009). Human immunodeficiency virus type 1 envelope gp120-induced partial T-cell receptor signaling creates an F-actin-depleted zone in the virological synapse. J Virol 83, 11341-11355.
39. Walker, L. M., Huber, M., Doores, K. J., Falkowska, E., Pejchal, R., Julien, J. P., Wang, S. K., Ramos, A., Chan-Hui, P. Y., Moyle, M., et al. (2011). Broad neutralization coverage of HIV by multiple highly potent antibodies. Nature 477, 466-470.
40. Yanagida, M., Suzuki, Y., and Toda, T. (1984). Molecular organization of the head of bacteriophage Teven: underlying design principles. Adv Biophys 17, 97-146.
41. Zhang, Z., Kottadiel, V. I., Vafabakhsh, R., Dai, L., Chemla, Y. R., Ha, T., and Rao, V. B. (2011). A promiscuous DNA packaging machine from bacteriophage T4. PLoS Biol 9, e1000592.
42. Reeves, P. J., Kim, J. M., and Khorana, H. G. (2002). Structure and function in rhodopsin: a tetracycline-inducible system in stable mammalian cell lines for high-level expression of opsin mutants. Proc Natl Acad Sci USA 99, 13413-13418.

All documents, patents, journal articles and other materials cited in the present application are incorporated herein by reference.

While the present disclosure has been disclosed with references to certain embodiments, numerous modifications, alterations, and changes to the described embodiments are possible without departing from the sphere and scope of the present disclosure, as defined in the appended claims. Accordingly, it is intended that the present disclosure not be limited to the described embodiments, but that it has the full scope defined by the language of the following claims, and equivalents thereof

What is claimed is:

1. An engineered viral particle comprising:
at least one viral vector; and
at least one CD4 ligand,
wherein the at least one CD4 ligand is displayed on the surface of the at least one viral vector.

2. The engineered viral particle of claim 1, wherein the at least one viral vector is selected from the group consisting of Lambda phage, Bacillus phage Phi29, Escherichia coli phages T2, T3, T4 and T7, Enterobacteriaphage P22, phage SPP1, Filamentous phages, Herpes viruses, adenoviruses, adeno-associated viruses (AAV), retroviruses, and lentiviruses.

3. The engineered viral particle of claim 2, wherein Filamentous phages are selected from the group consisting of M13, fd, and Fl.

4. The engineered viral particle of claim 1, wherein the CD4 ligand is an HIV component that interact with CD4.

5. The engineered viral particle of claim 1, wherein the CD4 ligand is CD4-binding DARPin (Designed Ankyrin Repeat Proteins) or HIV envelop protein.

6. The engineered viral particle of claim 1, wherein the CD4 ligand is linked to at least one protein selected from the group consisting of Hoc and Soc through a linker.

7. The engineered viral particle of claim 6, wherein the linker is a sequence consisting of 2-25 amino acids.

8. The engineered viral particle of claim 6, wherein the linker projects the CD4 ligand away at least 170 Å distance from the capsid wall of the viral vector.

9. The engineered viral particle of claim 6, wherein the linker is at least one selected from the group consisting of SEQ ID No. 1 and SEQ ID No. 2.

10. The engineered viral particle of claim 1, further comprise at least one nucleic acid packed in the viral vector.

11. An engineered viral particle comprising:
at least one viral vector; and
at least one HIV-1 envelope protein,
wherein the at least one HIV-1 envelope protein is displayed on the surface of the at least one viral vector.

12. The engineered viral particle of claim 11, wherein the HIV-1 envelope protein is selected from the group consisting of gp140, gp120, and a fragment of the envelope protein.

13. A method of activating a latent HIV-1 proviral genome comprising:
adding an engineered viral particle of claim 1 to T cells containing latent HIV-1 proviral genome; and
binding the engineered viral particle of claim 1 with T cells containing latent HIV-1 proviral genome.

14. The method of claim 13, wherein the engineered viral particle does not induce global activation of T cells.

15. The method of claim 13, wherein the engineered viral particle induces transcription of the HIV-1 proviral genome in T cells.
